(19) United States
(12) Patent Application Publication
(10) Pub. No.: US 2025/0171511 A1
(43) Pub. Date: May 29, 2025

(54) ANTIVIRAL PEPTIDES AND METHODS OF USE THEREOF

(71) Applicants: Versitech Limited, Hong Kong (CN); Centre for Virology, Vaccinology and Therapeutics Limited, Hong Kong (CN)

(72) Inventors: Hanjun Zhao, Hong Kong (CN); Kwok-Yung Yuen, Hong Kong (CN)

(21) Appl. No.: 18/724,569

(22) PCT Filed: Dec. 26, 2022
(86) PCT No.: PCT/CN2022/141996

§ 371 Date: Jun. 26, 2024

Related U.S. Application Data
(60) Provisional application No. 63/293,995, filed on Dec. 27, 2021.

(51) Int. Cl. C07K 14/47; A61K 9/00; A61K 38/17; A61K 45/06; A61P 31/14; A61P 31/16

(52) U.S. Cl. CPC C07K 14/4723; A61K 9/0073; A61K 38/1729; A61K 45/06; A61P 31/14; A61P 31/16

(57) ABSTRACT

Antiviral peptides and formulations thereof are described for use in treating or preventing one or more symptoms of coronavirus infections. Peptides derived from human beta defensin 2 have been shown to have antiviral properties against different variants of coronavirus including cross-linking viral particles, blocking cell-to-cell fusion, and/or inhibiting viral release. Pharmaceutical compositions and methods of using one or more antiviral peptides are also provided. Preferably, the antiviral peptides are administered via intranasal route to prevent or alleviate one or more symptoms of coronavirus infections such as reducing the syncytial formation and lung damage.

### FIELD OF THE INVENTION

The invention is generally directed to peptides having antiviral properties, and more particularly compositions and methods for preventing or treating infections caused by coronavirus variants.

### BACKGROUND OF THE INVENTION

Coronaviruses have caused three outbreaks (2003-SARS-CoV, 2012-MERS-CoV, and 2019-SARS-CoV-2) in the past twenty years. The SARS-CoV-2 pandemic has lasted for almost 2 years at the time of writing (J. F. Chan, et al., Lancet, 395 (2020) 514-523). However, there are few widely available drugs, which could effectively protect humans from SARS-CoV-2 infection. Patients with severe COVID-19 pneumonia have diffuse alveolar damage with syncytia formation in their lung tissue, which is attributed to viral spike-ACE2 mediated cell fusion (R. Bussani, et al., EBioMedicine, 61 (2020) 103104; Z. Xu, et al., Lancet Respir Med, 8 (2020) 420-422). The syncytia caused by viral spike-ACE2 mediated fusion might be related to the excessive inflammatory responses in severe COVID-19 patients and anti-syncytia drugs might yield better clinical outcomes (H. Ma, et al., Cell Discov, 7 (2021) 73; J. Buchrieser, et al., Embo j, 39 (2020) e106267; and L. Lin, et al., Cell Death Differ, 28 (2021) 2019-2021).

Effective antivirals and neutralizing antibodies, which block viral entry into susceptible host cells, can abort the first step of the viral replication cycle. Many studies have tried to identify viral entry inhibitors as the key antiviral strategy (R. Heida, et al., Drug Discov Today, 26 (2021) 122-137). SARS-CoV-2 is known to enter cells by binding to heparan sulfate (HS) and the receptor ACE2 which allows cell entry through TMPRSS2-mediated cell membrane fusion pathway or endocytosis pathway (T. M. Clausen, et al., Cell, 183 (2020) 1043-1057.e1015; and M. Hoffmann, et al., Cell, 181(2020) 271-280 e278). Though many studies have shown that some antivirals could block SARS-CoV-2 entry or viral RNA synthesis (D. Asarnow, et al., Cell, 184 (2021) 3192-3204.e3116; V. Gil Martínez, et al., Pharmaceuticals (Basel), 2021 Jul. 28;14(8):736; Y. W. Zhou, et al., Signal Transduct Target Ther, 6 (2021) 317), reports on how SARS-CoV-2 is released from infected cells to enter other uninfected cells are limited. Moreover, no antiviral has yet been clearly demonstrated to inhibit SARS-CoV-2 release.

There is a need for developing effective prophylactic and/or therapeutic therapy for those having or at risk of exposure to one or more respiratory pathogens such as SARS-CoV-2. Therefore, it is an object of the invention to provide prophylactic and/or therapeutic therapy for treating and/or preventing SARS-CoV-2 infections.

It is another object of the invention to provide compositions and methods for treating and/or preventing one or more of the pathological processes associated with SARS-CoV-2 infections.

### SUMMARY OF THE INVENTION

It has been discovered that antiviral peptides derived from human beta defensin 2 peptide broadly inhibited SARS-CoV-2 variants in vitro and in vivo.

Compositions including an antiviral peptide are described. The antiviral peptide generally has an amino acid sequence of SEQ ID NO:1, or a fragment or variant thereof, for example, in some cases the antiviral peptide has a sequence similarity of about 80%, 85%, 90%, 95%, 99% to SEQ ID NO: 1. In some forms, the antiviral peptide has an amino acid sequence of any one of SEQ ID NO:2, SEQ ID NO:3, or SEQ ID NO:4, or a variant thereof having a sequence similarity of about 80%, 85%, 90%, 95% to any one of SEQ ID NO: 2, SEQ ID NO:3, or SEQ ID NO:4. In some forms, the antiviral peptide has an amino acids sequence of SEQ ID NO:4. In some forms, the antiviral peptide has an amino acids sequence of SEQ ID NO:4 cross-linked in a form of tetramers. In some forms, two or more of the antiviral peptides are in a form of dimers, trimers, tetramers, or multimers.

In some forms, the antiviral peptide comprises an amino acid sequence that has the sequence SEQ ID NO: 1 or is a fragment or variant of SEQ ID NO:1. In some forms, the amino acid sequence has a sequence similarity of about 80%, 85%, 90%, 95%, 99% to SEQ ID NO:1. In some forms, the amino acid sequence has the sequence of any one of SEQ ID NO:2, SEQ ID NO:3, or SEQ ID NO:4, or is a variant of any one of SEQ ID NO: 2, SEQ ID NO:3, or SEQ ID NO:4. In some forms, the amino acid has a sequence similarity of about 80%, 85%, 90%, 95% to any one of SEQ ID NO:2, SEQ ID NO:3, or SEQ ID NO:4. In some forms, the amino acid sequence has the sequence of SEQ ID NO:4.

In some forms, the antiviral peptide has the sequence SEQ ID NO:1 or is a fragment or variant of SEQ ID NO:1. In some forms, the antiviral protein has a sequence similarity of about 80%, 85%, 90%, 95%, 99% to SEQ ID NO:1. In some forms, the antiviral peptide has the sequence of any one of SEQ ID NO:2, SEQ ID NO:3, or SEQ ID NO: 4, or is a variant of any one of SEQ ID NO:2, SEQ ID NO:3, or SEQ ID NO:4. In some forms, the antiviral peptide has a sequence similarity of about 80%, 85%, 90%, 95% to any one of SEQ ID NO:2, SEQ ID NO:3, or SEQ ID NO:4. In some forms, the antiviral peptide comprises SEQ ID NO:4.

A multimer is also provided, which has two or more antiviral peptides, wherein the antiviral peptides comprise an amino acid sequence that has the sequence SEQ ID NO:1 or is a fragment or variant of SEQ ID NO:1. In some embodiments, the amino acid sequence has a sequence similarity of about 80%, 85%, 90%, 95%, 99% to SEQ ID NO:1. In some embodiments, the amino acid sequence has the sequence of any one of SEQ ID NO:4, SEQ ID NO:2 or SEQ ID NO:3, or is a variant of any one of SEQ ID NO:4, SEQ ID NO:2 or SEQ ID NO:3. In some embodiments, the amino acid sequence has a sequence similarity of about 80%, 85%, 90%, 95% to any one of SEQ ID NO:4, SEQ ID NO:2, or SEQ ID NO:3. In some embodiments, the multimer is a dimer, trimer or tetramer, and/or is homomultimeric or heteromultimeric. In some embodiments, the multimer is formed by cross-linking each monomeric antiviral peptide, preferably using 2,2-bis(hydroxymethyl)propionic acid (MPA) or a 2nd generation MPA dendron with 4 reactive sites. In some embodiments, the multimer is formed by coupling a MPA to two monomeric antiviral peptides having SEQ ID NO:4 cross-linked by lysine at C terminal to form a molecule with two branches of the antiviral peptide or by coupling a 2nd generation MPA dendron with 4 reactive sites to monomeric antiviral peptide having SEQ ID NO: 4 cross-linked by lysine at C terminal to form a molecule with four branches of the antiviral peptide.

Pharmaceutical compositions including the antiviral peptide and a pharmaceutically acceptable carrier are also provided. In some embodiments, the pharmaceutical composition comprises the antiviral peptide or the multimer as described herein. In some forms, the pharmaceutical composition is lyophilized, or in the form of liquid or powder. Kits including the antiviral peptide are also described. Typically, the kids include one or more single unit dose of the antiviral peptide and instructions on how the dose is to be administered for treatment or prevent of coronavirus infection, influenza virus infection or rhinovirus infection. The influenza virus may be HIN1 virus and/or the rhinovirus may be HRV-1B or HRV-B14.

Also provided are methods of treating, retarding development of, or preventing development of one or more symptoms of respiratory viral infections or blocking virus transmission by administering to a subject in need thereof an effective amount of the antiviral peptide or the multimer or the antiviral peptide composition. The methods are particularly suited for use in a subject is having a respiratory viral infection or at risk of contracting a respiratory virus. In some forms, the respiratory virus is SARS-CoV-2 virus or a variant thereof, for example, SARS-CoV-2 B.1.1.7 (Alpha variant), SARS-CoV-2 B.1.351 (Beta variant), SARS-CoV-2 P.1 (Gamma variant), SARS-CoV-2 B.1.617, SARS-CoV-2 B.1.617.1 (Kappa variant), SARS-CoV-2 B.1.621 (Mu variant), SARS-CoV-2 B.1.617.2 (Delta variant), SARS-CoV-2 B.1.617.3, or SARS-CoV-2 B.1.1.529 (Omicron variant). The methods generally administer the composition to the pulmonary or nasal system, for example, in a form of powder, liquids, or suspensions. In some forms, the composition is administered in a form of aerosol, or via a nebulizer or an inhaler. In other forms, the composition is administered in combination with another therapeutic, prophylactic, or diagnostic agent. Exemplary agents include bronchodilators, corticosteroids, methylxanthines, phosphodiesterase-4 inhibitors, anti-angiogenesis agents, antimicrobial agents, antioxidants, anti-inflammatory agents, immunosuppressant agents, anti-allergic agents, and combinations thereof.

In some forms, the composition is administered at an interval selected from the group consisting of once a week, once every two weeks, approximately once a month, once every two months and once every three months, optionally once a week for up to a period of 1, 2, 3, 4, 5, or 6 months. In some forms, the composition is administered to a human subject at a dose of between 0.001 mg/kg body weight of the subject and 100 mg/kg body weight of the subject, inclusive; or at a dose of between 2.0 mg and 20 mg, inclusive; optionally at a dose of 5 mg. Preferably, the methods administer the composition in an amount effective to reduce syncytial formation and lung damage in the subject, optionally reduce one or more symptoms of cough, fatigue, fever, body aches, headache, sore throat, loss or altered sense of taste and/or smell, vomiting, diarrhea, cytokine storm, skin changes, ocular complications, confusion, chronic neurological impairment, chest pain and shortness of breath.

The antiviral peptide, the multimer or the composition as described herein for use in treating or retarding the development of one or more symptoms of respiratory viral infections or blocking virus transmission is provided which comprises administering to a subject in need thereof an effective amount of the antiviral peptide, the multimer or the composition. In some embodiment, the subject is having a respiratory viral infection or at risk of contracting a respiratory virus. In some embodiment, the respiratory virus is selected from a group consisting of SARS-CoV-2 virus, influenza virus or rhinovirus. In some embodiment, the influenza virus is HIN1 virus and/or the rhinovirus is HRV-1B or HRV-B14. In some embodiment, the virus is a SARS-CoV-2 virus variant selected from the group consisting of SARS-CoV-2 B.1.1.7 (Alpha variant), SARS-CoV-2 B.1.351 (Beta variant), SARS-CoV-2 P.1 (Gamma variant), SARS-CoV-2 B.1.617, SARS-CoV-2 B.1.617.1 (Kappa variant), SARS-CoV-2 B.1.621 (Mu variant), SARS-CoV-2 B.1.617.2 (Delta variant), SARS-CoV-2 B.1.617.3, and SARS-CoV-2 B.1.1.529 (Omicron variant). In some embodiment, the composition is administered to the pulmonary or nasal system. In some embodiment, the composition is administered in a form selected from the group consisting of powder, liquids, and suspensions. In some embodiment, the composition is administered in a form of aerosol. In some embodiment, the composition is administered via a nebulizer or an inhaler. In some embodiment, the composition is administered in combination with another therapeutic, prophylactic, or diagnostic agent. In some embodiment, the composition is administered in combination with one or more agents selected from the group consisting of bronchodilators, corticosteroids, methylxanthines, phosphodiesterase-4 inhibitors, anti-angiogenesis agents, antimicrobial agents, antioxidants, anti-inflammatory agents, immunosuppressant agents, anti-allergic agents, and combinations thereof. In some embodiment, the composition is administered at an interval selected from the group consisting of once a week, once every two weeks, approximately once a month, once every two months and once every three months. In some embodiment, the composition is administered once a week for up to a period of 1, 2, 3, 4, 5, or 6 months. In some embodiment, the composition is administered to a human subject at a dose of between 0.001 mg/kg body weight of the subject and 100 mg/kg body weight of the subject, inclusive. In some embodiment, the composition is administered to a human subject at a dose of between 2.0 mg and 20 mg, inclusive. In some embodiment, the composition is administered to the subject at a dose of 5 mg. In some embodiment, the composition is administered in an amount effective to reduce syncytial formation and lung damage in the subject. In some embodiment, the composition is administered in an amount effective to reduce one or more symptoms of cough, fatigue, fever, body aches, headache, sore throat, loss or altered sense of taste and/or smell, vomiting, diarrhea, cytokine storm, skin changes, ocular complications, confusion, chronic neurological impairment, chest pain and shortness of breath.

Use of the antiviral peptide, the multimer or the composition as described herein for the manufacture of a medicament or kit for treating or retarding the development of one or more symptoms of respiratory viral infections or blocking virus transmission is provided. In some embodiment, the subject is having a respiratory viral infection or at risk of contracting a respiratory virus. In some embodiment, the symptom is selected from a group consisting of cough, fatigue, fever, body aches, headache, sore throat, loss or altered sense of taste and/or smell, vomiting, diarrhea, cytokine storm, skin changes, ocular complications, confusion, chronic neurological impairment, chest pain and shortness of breath. In some embodiment, the respiratory virus is selected from a group consisting of SARS-CoV-2 virus, influenza virus or rhinovirus. In some embodiment, the influenza virus is HIN1 virus and/or the rhinovirus is HRV-1B or HRV-B14. In some embodiment, the virus is a SARS-CoV-2 virus variant selected from the group consisting of SARS-CoV-2 B.1.1.7 (Alpha variant), SARS-CoV-2 B.1.351 (Beta variant), SARS-CoV-2 P.1 (Gamma variant), SARS-CoV-2 B.1.617, SARS-CoV-2 B.1.617.1 (Kappa variant), SARS-CoV-2 B.1.621 (Mu variant), SARS-CoV-2 B.1.617.2 (Delta variant), SARS-CoV-2 B.1.617.3, and SARS-CoV-2 B.1.1.529 (Omicron variant). In some embodiment, the composition is for administration to the pulmonary or nasal system. In some embodiment, the composition is in a form selected from the group consisting of powder, liquids, and suspensions. In some embodiment, the composition is in a form of aerosol. In some embodiment, the composition is for administration via a nebulizer or an inhaler. In some embodiment, the medicament or kit comprises another therapeutic, prophylactic, or diagnostic agent. In some embodiment, the medicament or kit comprises one or more agents selected from the group consisting of bronchodilators, corticosteroids, methylxanthines, phosphodiesterase-4 inhibitors, anti-angiogenesis agents, antimicrobial agents, antioxidants, anti-inflammatory agents, immunosuppressant agents, anti-allergic agents, and combinations thereof. In some embodiment, the composition is administered at an interval selected from the group consisting of once a week, once every two weeks, approximately once a month, once every two months and once every three months. In some embodiment, the composition is administered once a week for up to a period of 1, 2, 3, 4, 5, or 6 months. In some embodiment, the composition is administered to a human subject at a dose of between 0.001 mg/kg body weight of the subject and 100 mg/kg body weight of the subject, inclusive. In some embodiment, the composition is administered to a human subject at a dose of between 2.0 mg and 20 mg, inclusive. In some embodiment, the composition is administered to the subject at a dose of 5 mg. In some embodiment, the composition is administered in an amount effective to reduce syncytial formation and lung damage in the subject. In some embodiment, the composition is administered in an amount effective to reduce one or more symptoms of cough, fatigue, fever, body aches, headache, sore throat, loss or altered sense of taste and/or smell, vomiting, diarrhea, cytokine storm, skin changes, ocular complications, confusion, chronic neurological impairment, chest pain and shortness of breath.

### BRIEF DESCRIPTION OF THE DRAWINGS

FIGS. 1A-1E are bar graphs showing antiviral activity against SARS-CoV-2 using plaque reduction assay. FIG. 1A is a bar graph showing percent of plaque forming units (PFU %) in the presence of H23, H26, H30 peptides at the concentrations of 25, 12.5, and 0 μg/ml in low salt condition (30 mM phosphate buffer, PBS/5) determined by plaque reduction assay (n=3); FIG. 1B is a bar graph showing percent of plaque forming units (PFU %) in the presence of H23, H26, H30 peptides at the concentrations of 25, 12.5, and 0 μg/ml in high salt condition (150 mM PBS) determined by plaque reduction assay (n=3); FIG. 1C is a bar graph showing in percent of plaque forming units (PFU %) in the presence of 2-branced H30 (2H30) and 4-branced H30 (4H30) peptides at the concentrations between 0 to 12.5 μg/ml in PBS determined by plaque reduction assay (n=4); FIG. 1D is a bar graph showing cell viability (%) of VeroE6 and Calu-3 cells in the presence of 4H30 peptide at the concentrations between 0 to 400 μg/ml (n=3); FIG. 1E is a bar graph showing percent of plaque forming units (PFU %) in the presence of DMEM or 4H30 against SARS-CoV-2 (B.1.1.63) in VeroE6 and Calu-3 cells (n=6) post-infection. After 6 h viral infection, 4H30 (50 μg ml⁻¹) was added to infected cells and viral titers in supernatants were measured at 24-30 h post-infection, * indicates P<0.05, ** indicates the P<0.01 when compared with DMEM, P values were calculated by the two-tailed Student's t test, Data are presented as mean±SD of indicated independent biological samples.

FIGS. 2A-2F are bar graphs. FIG. 2A shows viral RNA copies (RNA copy, 0-16 fold) for each of samples pretreated with 4H30 (25 μg ml⁻¹) 1 h before viral infection (1 h-Pre), relative to mock-treated virus (Mock) and virus pretreated with 4H30 before viral infection (Pre-mix), respectively, measured at 1 hour (1 h) post infection. Relative RNA copies (fold) were normalized to mock-treated virus (n=4); FIG. 2B shows viral RNA copies (viral RNA copy to 1 h, 0-90 fold) for each of samples pretreated with 4H30 (25 μg ml⁻¹) 1 h before viral infection (1 h-Pre), relative to mock-treated virus (Mock) and virus pretreated with 4H30 before viral infection (Pre-mix), respectively, measured at 6 hours (6 h) post infection (n=4); FIG. 2C shows viral attachment, showing RNA copies measured by RT-qPCR (Relative RNA, 0-5 fold) for SARS-CoV-2 samples treated with each of DMEM, 25 μg ml⁻¹ 4H30 (4H30), 0.1% Triton X100 (Triton), neutralizing antibody (Anti-S) or Mock then was added to VeroE6 cells for attachment at 4° C. (n=4), * indicates P<0.05, ** indicates the P<0.01 when compared with DMEM. P values were calculated by the two-tailed Student's t test, Data are presented as mean±SD of indicated four independent biological samples; FIG. 2D shows 4H30 binding to spike, compared with ACE2 (n=8), showing OD value (0-3.0) of Spike(S), and S-bound 4H30 at 150 ng, 37.5 ng, or 0 ng, or ACE2 (100 ng) bound to coated 4H30 at 150 ng, 37.5 ng, or 0 ng, and ACE2 alone, respectively, on ELISA plate, * indicates P<0.05 when compared with ACE2; FIG. 2E shows binding to spike (100 ng) bound to coated ACE2 on ELISA plate, showing relative binding to ACE2 (0-125%) for each of Spike (S), spike treated with antibody (Ab), ACE2 treated 4H30 (4H30), and PBS alone (Mock), respectively, (n=4). Spike treated with neutralizing antibody (Ab) was used as the control, ** indicates P<0.01 when compared with S+PBS; FIG. 2F shows 4H30 activity against SARS-CoV-2 virus pretreated with 4H30 (50 μg ml⁻¹), showing PFU/ml (10⁴-10⁷) for each of PBS, 4H30 and Mock, respectively, (n=4), * indicates P<0.05 when compared with PBS.

FIGS. 3A to 3J are bar graphs. FIG. 3A shows viral RNA copy (0-120%) for each of samples including DMED, 4H30-6h, and Mock, respectively, for 10-hpi supernatant; FIG. 3B shows viral RNA copy (10⁶-10¹⁰) for each of samples treated with DMEM, 4H30 or Mock (cells without infection), at 10 h post-infection; FIG. 3C shows viral RNA copy (0-200%) for each of samples including 14 h, BSA, 4H30, CS, HS, 4H30-CS, 4H30-HS, and 4H30-BSA, respectively, for 18-hpi supernatant; FIG. 3D shows OD value (0-0.40) for each of CS and HS samples in the presence of 300 ng GAG, 75 ng GAG and 0 ng GAG, as well as 4H30, respectively, for 4H30 binding; FIG. 3E shows viral RNA copy (0-1000%) for each of samples including DMEM, 4H30, BSA, CS, HS, 4H30-BSA, 4H30-CS, 4H30-HS, and Mock respectively, for A549; FIG. 3F shows SARS-CoV-2 attachment to Calu-3 cells at 4d following 4H340 treatment, with viral RNA copies/well (10⁵-10⁸) for each of samples treated with DMEM, 4H30 or Mock, respectively; * indicates P<0.05 when compared with DMEM. P values were calculated by the two-tailed Student's t test. Data are presented as mean±SD of indicated four independent biological samples; FIG. 3G shows viral RNA copy (0-250%) for each of samples including DMEM or 4H30 in the presence of Mock, 4H30, and ChABC+Hase, respectively, for A549; FIG. 3H is a graph of SARS-CoV-2 replication in Calu-3 cells pretreated by 4H30 for 1 h and then exposed to SARS-CoV-2, showing PFU/ml (10¹-10⁵) for each of samples treated with DMEM, 4H30 or Mock (Cells without infection), respectively (n=4). All data are presented as mean±SD of four independent biological samples; FIG. 3I shows viral RNA copy (0-350%) for each of samples including DMEM, 4H30, CS, HS, 4H30-CS, 4H30-HS, and 4H30-BSA, respectively; respectively; FIG. 3J shows viral RNA copy (0-140%) for each of CS and HS samples in the presence of 320 ng GAG, 80 ng GAG and 0 ng 4H30 (12.5 μg/ml GAG), as well as, DMEM, respectively.

FIGS. 4A-4E are bar graphs. FIG. 4A is a line graph of inhibited endosomal acidification in VeroE6 cells, showing change in body weight (50%-130%) over time (0-40 Days) for each of 4H30 and Mock, respectively; FIG. 4B shows PFU/lung (10⁵-10⁸) for each of samples including 4H30-24 h, 4H30-8 h, 4H30-4 h, 4H30-Pre, Mock and H23-Pre, respectively, for B.1.1.63; FIG. 4C shows PFU/lung (10⁵-10⁸) for each of Mock, 4H30-0.5 and 4H30-0.1, respectively, for B.1.1.63; FIG. 4D shows PFU (0-140%) for each of samples including B.1.163 (IC₅₀=0.48), B.1.1.7 (IC₅₀=0.75), B.1.351 (IC₅₀=0.89), and B.1.617.2 (IC₅₀=0.79), respectively; FIG. 4E shows PFU/lung (10⁴-10⁹) for each of Mock, 4H30 and Remde, respectively, for B.1.617.2 Post exposure; P=0.061.

FIG. 5 is a bar graph showing the inhibitory effect of 4H30 on H1N1 virus replication, data are presented as mean±SD of at least three biological samples with more than two independent experiments.

FIG. 6 is a bar graph showing that 4H30 inhibits minor group HRV-1B and major group HRV-B14 using plaque reduction assay, viral RNA copy (%) was the viral RNA of 4H30-treated virus normalized to that of untreated virus (0 μg/ml peptide), data are presented as mean±SD of at least three independent biological samples.

### DETAILED DESCRIPTION OF THE INVENTION

### I. Definitions

The terms “individual”, “host”, “subject”, and “patient” are used interchangeably, and refer to a mammal, including, but not limited to, murines, simians, humans, mammalian farm animals, mammalian sport animals, and mammalian pets.

The term “effective amount” or “therapeutically effective amount” refers to the amount which is able to treat one or more symptoms of a disease or disorder, reverse the progression of one or more symptoms of a disease or disorder, halt the progression of one or more symptoms of a disease or disorder, or prevent the occurrence of one or more symptoms of a disease or disorder in a subject to whom the formulation is administered, for example, as compared to a matched subject not receiving the compound. The actual effective amounts of compound can vary according to the specific compound or combination thereof being utilized, the particular composition formulated, the mode of administration, and the age, weight, condition of the individual, and severity of the symptoms or condition being treated.

The term “pharmaceutically acceptable” or “biocompatible” refers to compositions, polymers, and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

The term “pharmaceutically acceptable salt” is art-recognized, and includes relatively non-toxic, inorganic and organic acid addition salts of compounds. Examples of pharmaceutically acceptable salts include those derived from mineral acids, such as hydrochloric acid and sulfuric acid, and those derived from organic acids, such as ethanesulfonic acid, benzenesulfonic acid, and p-toluenesulfonic acid. Examples of suitable inorganic bases for the formation of salts include the hydroxides, carbonates, and bicarbonates of ammonia, sodium, lithium, potassium, calcium, magnesium, aluminum, and zinc. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; etc.

The terms “inhibit” or “reduce” in the context of inhibition, mean to reduce or decrease in activity and quantity. This can be a complete inhibition or reduction in activity or quantity, or a partial inhibition or reduction. Inhibition or reduction can be compared to a control or to a standard level. Inhibition can be measured as a % value, e.g., from 1% up to 100%, such as 5%, 10, 25, 50, 75, 80, 85, 90, 95, 99, or 100%. For example, compositions including therapeutic agents may inhibit or reduce one or more markers of a disease or disorder in a subject by about 10%, 20%, 30%, 40%, 50%, 75%, 85%, 90%, 95%, or 99% from the activity and/or quantity of the same marker in subjects that did not receive, or were not treated with the compositions. In some forms, the inhibition and reduction are compared according to the level of mRNAs, proteins, cells, tissues and organs.

The terms “treating” or “retarding development of” in the context of a disease or disorder mean to ameliorate, reduce or otherwise stop a disease, disorder or condition from occurring or progressing in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating, or palliating the disease state, and remission or improved prognosis. For example, an individual is successfully “treated” if one or more symptoms associated with a coronavirus infection are mitigated or eliminated, including, but are not limited to, reducing and/or inhibiting the syncytial formation and lung damage, increasing the quality of life of those suffering from the disease, decreasing the dose of other medications required to treat the disease, delaying the progression of the disease, and/or prolonging survival of individuals.

The term “biodegradable” generally refers to a material that will degrade or erode under physiologic conditions to smaller units or chemical species that are capable of being metabolized, eliminated, or excreted by the subject. The degradation time is a function of composition and morphology.

The terms “protein” or “polypeptide” or “peptide” refer to any chain of more than two natural or unnatural amino acids, regardless of post-translational modification (e.g., glycosylation or phosphorylation), constituting all or part of a naturally occurring or non-naturally occurring polypeptide or peptide.

The terms “Coronavirus disease 2019”, “COVID-19”, or “COVID” refer to the disease caused by the human pandemic SARS-CoV-2 virus.

The term “combination therapy” refers to treatment of a disease or symptom thereof, or a method for achieving a desired physiological change, including administering to an animal, such as a mammal, especially a human being, an effective amount of two or more chemical agents or components to treat the disease or symptom thereof, or to produce the physiological change, wherein the chemical agents or components are administered together, such as part of the same composition, or administered separately and independently at the same time or at different times (i.e., administration of each agent or component is separated by a finite period of time from each other).

The term “dosage regime” refers to drug administration regarding formulation, route of administration, drug dose, dosing interval and treatment duration.

Recitation of ranges of values herein are merely intended to serve as a shorthand method of referring individually to each separate value falling within the range, unless otherwise indicated herein, and each separate value is incorporated into the specification as if it were individually recited herein.

### II. Compositions

It has been established that identify antiviral peptides derived from human beta defensin 2 peptide broadly inhibited SARS-CoV-2 variants in vitro and in vivo. Mechanistic studies in the Examples show that such antiviral peptides cross-linked SARS-CoV-2 to form viral clusters which failed to enter VeroE6 and Calu-3 cells. The cross-linking mechanism of the antiviral peptides effectively blocked both entry pathways of SARS-CoV-2 (namely the endocytic pathway and TMPRSS2-mediated entry pathway). Moreover, the antiviral peptides inhibited endosomal acidification to block spike-ACE2 mediated fusion through endocytosis. Thus, escaped viral particles which were not cross-linked and entered the endosomes would be stopped at this step of the viral replication cycle. Furthermore, the inhibition by the antiviral peptides on spike-ACE2 mediated fusion could reduce the syncytia formation which may reduce the pathology in vivo. Finally, the antiviral peptides inhibited SARS-CoV-2 release by cross-linking SARS-CoV-2 spike with glycosaminoglycans (GAGs), which significantly restricted SARS-CoV-2release as the last strategy to terminate the viral replication cycle. Such human antiviral peptides with triple antiviral mechanism can create a protective barricade on cell surface to block both entry and release of SARS-CoV-2. Importantly, inhaled antiviral peptides provided pre-exposure and post-exposure activity against SARS-CoV-2 variants and reduce syncytia formation in lungs of challenged hamsters. The finding underscores the clinical relevance in the development of this human-sourced peptide for treating COVID-19. Thus, in some forms, the composition includes one or more antiviral peptides derived from HBD2. In preferred forms, the composition includes a single antiviral peptide derived from HBD2a capable of inhibiting viral entry, fusion, and release of one or more respiratory viruses.

The human beta defensin 2 (HBD2) amino acid sequence (UniProtKB-O15263 (DFB4A_HUMAN)) is as follows:

(SEQ ID NO: 1)
MRVLYLLFSF LFIFLMPLPG VFGGIGDPVT CLKSGAICHP
VFCPRRYKQI GTCGLPGTKC CKKP

Compositions of antiviral peptides are provided for administration to a subject. Disclosed are HBD2 protein variants, compositions comprising such variants, and methods of using the variants and compositions. In some forms, the composition includes HBD2 variants at least about 50%, 55%, 60%, 65%, 70%, 75%, 80%, 85%, 90%, to 95% identical to the amino acid sequence of SEQ ID NO:1.

A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides disclosed herein and still obtain a molecule having similar characteristics as the polypeptide (e.g., a conservative amino acid substitution). For example, certain amino acids can be substituted for other amino acids in a sequence, without appreciable loss of activity. Since it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, and antigens. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly when the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

Amino acid substitutions are generally based on the relative similarity of the amino acid side-chain substituents, for example, their hydrophobicity, hydrophilicity, charge, and size. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). The polypeptides can include variants having about 50%, 60%, 70%, 80%, 90%, and 95% sequence identity to the polypeptide of interest.

“Identity” and “similarity” can be readily calculated by known methods, such as those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo and Lipman, SIAM J Applied Math, 48:1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48:443-453, 1970) algorithm (e.g., NBLAST, and XBLAST). The default parameters are used to determine the identity for the polypeptides of the present disclosure.

By way of example, a polypeptide sequence may be identical to the reference sequence, that is be 100% identical, or it may include up to a certain integer number of amino acid alterations as compared to the reference sequence such that the % identity is less than 100%. Such alterations include at least one amino acid deletion, substitution, including conservative and non-conservative substitution, or insertion, wherein the alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from the total number of amino acids in the reference polypeptide.

### A. Antiviral Peptides

In some forms, the antiviral peptides are derived from HBD2 or a fragment of HBD2. Exemplary antiviral peptides are shown in Table 1. In some forms, the antiviral peptides are one or more variants of SEQ ID NO:2, SEQ ID NO:3, or SEQ ID NO:4. In some forms, the antiviral peptides have at least about 80%, 85%, 90%, 95%, or 99% identical to the amino acid sequence of SEQ ID NO:2, SEQ ID NO:3, or SEQ ID NO:4. In some forms, the antiviral peptide is of SEQ ID NO:2, SEQ ID NO:3, or SEQ ID NO:4.

In some forms, the antiviral peptide variants can include one or more substitution mutations. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

TABLE 1
H23, H26, H30, 2H30, and 4H30 peptides

| Peptide | Sequence |
|---|---|
| H23 | CHPVFCPRRYKQIGTCGLPGTKC |
| | (SEQ ID NO: 2) |
| H26 | CHPVFCPRRYKQIGTCGLPGTKCCKK |
| | (SEQ ID NO: 3) |
| H30 | GAICHPVFCPRRYKQIGTCGLPGTKCCKKP |
| | (SEQ ID NO: 4) |
| 2H30 | GAICHPVFCPRRYKQIGTCGLPGTKCCKKP-2MPA |
| 4H30 | GAICHPVFCPRRYKQIGTCGLPGTKCCKKP-4MPA |

### 1. Modifications of Antiviral Peptides

In some forms, the antiviral peptides are modified to improve their pharmacokinetic and pharmacodynamic profiles. In some forms, the active antiviral peptides are monomers. In some forms, the antiviral peptides are dimerized, trimerized, tetramerized or multimerized. Dimerization, trimerization, tetramerization, or multimerization can occur between or among two or more antiviral peptides (same or different) through dimerization, trimerization, tetramerization, or multimerization domains. Alternatively, dimerization, trimerization, tetramerization, or multimerization of antiviral peptides can occur by chemical crosslinking. The dimers, trimers, tetramers, or multimers that are formed can be homodimeric/homomultimeric or heterodimeric/heteromultimeric.

The term “monomer” refers to a single antiviral peptide molecule. The terms “dimers”, “trimers”, “tetramers”, or “multimers” refer to two, three, four, or more monomers, respectively, forming one polypeptide molecule. The dimers, trimers, tetramers, or multimers may be homodimers, homotrimers, homotetramers, or homomultimers containing the same amino acid sequences for each of the monomers forming the dimers, trimers, tetramers, or multimers. The dimers, trimers, tetramers, or multimers may be heterodimers, heterotrimers, heterotetramers, or heteromultimers containing different amino acid sequences for each of the monomers forming the dimers, trimers, tetramers, or multimers.

In preferred forms, the antiviral peptides are dimers or tetramers formed by cross-linking each monomeric antiviral peptide using 2,2-bis(hydroxymethyl)propionic acid (MPA). In one form, it is a MPA which is coupled to two monomeric antiviral peptides having SEQ ID NO:4 cross-linked by lysine at C terminal to form a molecule with two branches of the antiviral peptide. In one form, it is a 2nd generation MPA dendron with 4reactive sites coupled to monomeric antiviral peptide having SEQ ID NO:4 cross-linked by lysine at C terminal to form a molecule with four branches of the antiviral peptide.

### Structure I: Chemical Formula of 2,2-bis(hydroxymethyl)propionic acid

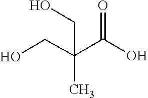

### B. Additional Therapeutic, Prophylactic or Diagnostic Agents

One or more additional therapeutic, diagnostic, and/or prophylactic agents may be used to treat or retard development of, or prevent development of inflammation in the lungs, and/or systemic inflammation resulting from COVID-19 induced pneumonia.

In addition to the antiviral peptides, the composition can contain one or more additional therapeutic, diagnostic, and/or prophylactic agents. In some forms, the composition may contain one or more additional compounds to relief symptoms such as inflammation, or shortness of breath. Representative therapeutic (including prodrugs), prophylactic, or diagnostic agents can be peptides, proteins, carbohydrates, nucleotides or oligonucleotides, small molecules, or combinations thereof. The active agents can be a small molecule active agent or a biomolecule, such as an enzyme or protein, polypeptide, or nucleic acid. Suitable small molecule active agents include organic and organometallic compounds. In some instances, the small molecule active agent has a molecular weight of less than about 2000 g/mol, more preferably less than about 1500 g/mol, most preferably less than about 1200 g/mol. The small molecule active agent can be a hydrophilic, hydrophobic, or amphiphilic compound. In some cases, one or more additional active agents may be dissolved or suspended in the pharmaceutically acceptable carrier.

In the case of pharmaceutical compositions for the treatment of lung diseases, the formulation may contain one or more therapeutic agents to treat, prevent or diagnose a disease or disorder of the lung. Non-limiting examples of therapeutic agents include bronchodilators, corticosteroids, methylxanthines, phosphodiesterase-4 inhibitors, anti-angiogenesis agents, antibiotics, antioxidants, anti-viral agents, anti-fungal agents, anti-inflammatory agents, immunosuppressant agents, anti-allergic agents, and combinations thereof.

The amount of a second therapeutic generally depends on the severity of lung disorders to be treated. Specific dosages can be readily determined by those of skill in the art. See Ansel, Howard C. et al. Pharmaceutical Dosage Forms and Drug Delivery Systems (6^{th} ed.) Williams and Wilkins, Malvern, PA (1995).

In other forms, one or more agents include bronchodilators, corticosteroids, methylxanthines, phosphodiesterase-4 inhibitors, anti-angiogenesis agents, antibiotics, antioxidants, anti-viral agents, anti-fungal agents, anti-inflammatory agents, immunosuppressant agents, and/or anti-allergic agents, are administered prior to, in conjunction with, subsequent to, or alternation with treatment with the disclosed antiviral peptide formulation.

The additive drug may be present in its neutral form, or in the form of a pharmaceutically acceptable salt. In some cases, it may be desirable to prepare a formulation containing a salt of an active agent due to one or more of the salt's advantageous physical properties, such as enhanced stability or a desirable solubility or dissolution profile.

In some forms, the additional agent is a diagnostic agent imaging or otherwise assessing the site of application. Exemplary diagnostic agents include paramagnetic molecules, fluorescent compounds, magnetic molecules, and radionuclides, x-ray imaging agents, and contrast media. These may also be ligands or antibodies which are labelled with the foregoing or bind to labelled ligands or antibodies which are detectable by methods known to those skilled in the art.

In certain forms, the pharmaceutical composition contains one or more local anesthetics. Representative local anesthetics include tetracaine, lidocaine, amethocaine, proparacaine, lignocaine, and bupivacaine. In some forms, one or more additional agents, such as a hyaluronidase enzyme, is also added to the formulation to accelerate and improves dispersal of the local anesthetic.

### 1. Bronchodilators

In some forms, antiviral peptides and formulations thereof are used in combination with one or more bronchodilators. Bronchodilators are a type of medication that helps open the airways to make breathing easier.

Short-acting bronchodilators in an emergency or as needed for quick relief. Some exemplary short-acting bronchodilators include anticholinergics such as ipratropium (e.g., ATROVENT®, in COMBIVENT®, in DUONEB®), beta2-agonists such as albuterol (e.g., VOSPIRE ER®, in COMBIVENT®, in DUONEB®), and levalbuterol (e.g., XOPENEX®).

Long-acting bronchodilators are used to treat COPD over an extended period of time. They are usually taken once or twice daily over a long period of time, and they come as formulations for inhalers or nebulizers. Some exemplary long-acting bronchodilators include anticholinergics such as aclidinium (e.g., TUDORZA®), tiotropium (e.g., SPIRIVA®), or umeclidinium (e.g., INCRUSE ELLIPTA®), beta2-agonists such as arformoterol (e.g., BROVANA®), formoterol (e.g., FORADIL®, PERFOROMIST®), indacaterol (e.g., ARCAPTA®), salmeterol (e.g., SEREVENT®), and olodaterol (e.g., STRIVERDI RESPIMAT®).

### 2. Corticosteroids

In some forms, antiviral peptides or formulations thereof are used in combination with one or more corticosteroids. Corticosteroids help reduce inflammation in the body, making air flow easier to the lungs. There are several corticosteroids. Some are prescribed with bronchodilators because these two medications can work together to make breathing more effective. Fluticasone (e.g., FLOVENT®), budesonide (e.g., PULMICORT®), and prednisolone are the ones doctors commonly prescribe for COPD.

### 3. Methylxanthines

Methylxanthines are heterocyclic compounds that are methylated derivatives of xanthine comprising of coupled pyrimidinedione and imidazole rings (Talik et al., Separ. Purif. Rev. 2012;41:1-61). Methylxanthines have been widely used for therapeutic purposes for decades, with proven therapeutic benefits in different medical scopes. For example, the naturally occurring methylxanthines like caffeine, theophylline, and theobromine have been used in the treatment of respiratory diseases (Lam and Newhouse, Chest. 1990;98:44-52), cardiovascular diseases, cancer (Hayashi et al., Anticancer Res. 2005;25:2399-2405; Kimura et al., J. Orthop. Sci. 2009;14:556-565) and the commercially produced xanthine derivative drug like pentoxifylline has been widely documented to have immunomodulatory properties.

In some forms, antiviral peptides and formulations thereof are used in combination with one or more methylxanthines such as pentoxifylline and caffeine. Potential beneficial properties of methylxanthines like pentoxifylline and caffeine as an adjuvant therapy to treat COVID-19 patients have been suggested (Monji F et al., Eur J Pharmacol. 2020 Nov. 15; 887: 173561). In these cases, theophylline (e.g., THEO-24®, THEOLAIR®, ELIXOPHYLLINE®, QUIBRON-T®, UNIPHYL®, and ELIXOPHYLLIN®), can be used, which works as an anti-inflammatory and/or antioxidant, and relaxes the muscles in the airway, to take along with a bronchodilator. Theophylline comes as a pill or a liquid to be taken on a daily basis, and/or combined with other medications.

### 4. Phosphodiesterase-4 Inhibitors

Benefits of roflumilast, a Phosphodiesterase-4 (PDE-4) inhibitor as a comprehensive support COVID-19 pathogenesis has been described (Sugin Lal Jabaris S et al., Pulm Pharmacol Ther. 2021 Feb; 66: 101978). Roflumilast, a well-known anti-inflammatory and immunomodulatory drug, is protective against respiratory models of chemical and smoke induced lung damage. There is significant data which demonstrate the protective effect of PDE-4 inhibitor in respiratory viral models and is likely to be beneficial in combating COVID-19 pathogenesis.

In some forms, antiviral peptides and formulations thereof are used in combination with one or more phosphodiesterase-4 inhibitors. In some forms, the compositions help relieve inflammation and/or improve air flow to the lungs. Several PDE-4 inhibitors have been identified such as cilomilast, piclamilast, oglemilast, tetomilast, tofimilast, ronomilast, revamilast, UK-500,001, AWD 12-281, CDP840, CI-1018, GSK256066, YM976, GS-5759 to treat chronic obstructive pulmonary disease (COPD) and asthma. CHF 6001, is an inhaled PDE-4 inhibitor currently undergoing phase II clinical trials for COPD. Also, two orally administered PDE-4 inhibitors such as roflumilast and apremilast have been approved in a row as treatments against inflammatory diseases including COPD, psoriasis, and psoriatic arthritis.

### 5. Antimicrobial Agents

In some forms, antiviral peptides or formulations thereof are used in combination with one or more antimicrobial agents. An antimicrobial agent is a substance that kills or inhibits the growth of microbes such as bacteria, fungi, viruses, or parasites. Antimicrobial agents include antiviral agents, antibacterial agents, antiparasitic agents, and anti-fungal agents. Representative antiviral agents include ganciclovir and acyclovir. Representative antibiotic agents include aminoglycosides such as streptomycin, amikacin, gentamicin, and tobramycin, ansamycins such as geldanamycin and herbimycin, carbacephems, carbapenems, cephalosporins, glycopeptides such as vancomycin, teicoplanin, and telavancin, lincosamides, lipopeptides such as daptomycin, macrolides such as azithromycin, clarithromycin, dirithromycin, and erythromycin, monobactams, nitrofurans, penicillins, polypeptides such as bacitracin, colistin and polymyxin B, quinolones, sulfonamides, and tetracyclines.

Other exemplary antimicrobial agents include iodine, silver compounds, moxifloxacin, ciprofloxacin, levofloxacin, cefazolin, tigecycline, gentamycin, ceftazidime, ofloxacin, gatifloxacin, amphotericin, voriconazole, natamycin.

### 6. Local Anesthetics

In some forms, antiviral peptides and formulations thereof are used in combination with one or more local anesthetics. A local anesthetic is a substance that causes reversible local anesthesia and has the effect of loss of the sensation of pain. Non-limiting examples of local anesthetics include ambucaine, amolanone, amylocaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethysoquin, dimethocaine, diperodon, dycyclonine, ecgonidine, ecgonine, ethyl chloride, etidocaine, beta-eucaine, euprocin, fenalcomine, formocaine, hexylcaine, hydroxytetracaine, isobutyl p-aminobenzoate, leucinocaine mesylate, levoxadrol, lidocaine, mepivacaine, meprylcaine, metabutoxycaine, methyl chloride, myrtecaine, naepaine, octacaine, orthocaine, oxethazaine, parethoxycaine, phenacaine, phenol, piperocaine, piridocaine, polidocanol, pramoxine, prilocaine, procaine, propanocaine, proparacaine, propipocaine, propoxycaine, psuedococaine, pyrrocaine, ropivacaine, salicyl alcohol, tetracaine, tolycaine, trimecaine, zolamine, and any combination thereof. In other aspects of this form, the antiviral peptides or formulations thereof include an anesthetic agent in an amount of, e.g., about 10 mg, about 50 mg, about 100 mg, about 200 mg, or more than 200 mg. The concentration of local anesthetics in the compositions can be therapeutically effective meaning the concentration is adequate to provide a therapeutic benefit without inflicting harm to the patient.

### 7. Anti-Inflammatory Agents

In some forms, antiviral peptides and formulations thereof are used in combination with one or more anti-inflammatory agents. Anti-inflammatory agents reduce inflammation and include steroidal and non-steroidal drugs. Suitable steroidal active agents include glucocorticoids, progestins, mineralocorticoids, and corticosteroids. Other exemplary anti-inflammatory agents include triamcinolone acetonide, fluocinolone acetonide, prednisolone, dexamethasone, loteprendol, fluorometholone, ibuprofen, aspirin, and naproxen. Exemplary immune-modulating drugs include cyclosporine, tacrolimus, and rapamycin. Exemplary non-steroidal anti-inflammatory drugs (NSAIDs) include mefenamic acid, aspirin, diflunisal, salsalate, ibuprofen, naproxen, fenoprofen, ketoprofen, deacketoprofen, flurbiprofen, oxaprozin, loxoprofen, indomethacin, sulindac, etodolac, ketorolac, diclofenac, nabumetone, piroxicam, meloxicam, tenoxicam, droxicam, lornoxicam, isoxicam, meclofenamic acid, flufenamic acid, tolfenamic acid, elecoxib, rofecoxib, valdecoxib, parecoxib, lumiracoxib, etoricoxib, firocoxib, sulphonanilides, nimesulide, niflumic acid, and licofelone.

In some forms, anti-inflammatory agents are anti-inflammatory cytokines. Exemplary cytokines are IL-10, TGF-β and IL-35.

### C. Formulations

Formulations of antiviral peptides are also provided. The antiviral peptides can be formulated for administration to a subject, for example, as a pharmaceutical formulation. Exemplary formulations include a solution, a dry powder, a tablet, micelles, colloids, nanodroplets, nano-structured hydrogel, nanocrystals, and a nanosuspension. Typically, the formulation includes a determined amount of antiviral peptides, in a form appropriate for a desired route of administration. The compositions can be stored lyophilized in single use vials for rehydration immediately before use. Other means for rehydration and administration are known to those skilled in the art.

Pharmaceutical formulations contain antiviral peptides in combination with one or more pharmaceutically acceptable excipients. Representative excipients include solvents, diluents, pH modifying agents, preservatives, antioxidants, suspending agents, wetting agents, viscosity modifiers, tonicity agents, stabilizing agents, and combinations thereof. Suitable pharmaceutically acceptable excipients are preferably selected from materials which are generally recognized as safe (GRAS), and may be administered to an individual without causing undesirable biological side effects or unwanted interactions.

Generally, pharmaceutically acceptable salts can be prepared by reaction of the free acid or base forms of an active agent with a stoichiometric amount of the appropriate base or acid in water or in an organic solvent, or in a mixture of the two; generally, non-aqueous media like ether, ethyl acetate, ethanol, isopropanol, or acetonitrile are preferred. Pharmaceutically acceptable salts include salts of an active agent derived from inorganic acids, organic acids, alkali metal salts, and alkaline earth metal salts as well as salts formed by reaction of the drug with a suitable organic ligand (e.g., quaternary ammonium salts). Lists of suitable salts are found, for example, in Remington's Pharmaceutical Sciences, 20th ed., Lippincott Williams & Wilkins, Baltimore, MD, 2000, p. 704.

Exemplary formulations of antiviral peptides include liquids and dry powders. In some forms, the antiviral peptides in an amount from about 1% to about 100%, inclusive, from about 1% to about 80%, from about 1% to about 50%, preferably from about 1% to about 40% by weight, more preferably from about 1% to about 20% by weight, most preferably from about 1% to about 10% by weight. The ranges above are inclusive of all values from 1% to 100%.

### 1. Dry Powder Formulations

In some forms, antiviral peptides are formulated in dry powder forms as finely divided solid formulations. The dry powder components can be stored in separate containers or mixed at specific ratios and stored. In some forms, suitable aqueous and organic solvents are included in additional containers. In other forms, dry powder components, one or more solvents, and instructions on procedures to mix and prepare assembled nanostructures are included in a kit. Alternatively, stabilized, assembled particles, nanoparticles or bulk gel thereof are dried via vacuum-drying or freeze-drying, and suitable pharmaceutical liquid carrier can be added to rehydrate and suspend the assembled nanostructures or gel compositions upon use.

Dry powder formulations are typically prepared by blending one or more gelators, stabilizing agents, or active agents with one or more pharmaceutically acceptable carriers. Pharmaceutical carrier may include one or more dispersing agents. The pharmaceutical carrier may also include one or more pH adjusters or buffers. Suitable buffers include organic salts prepared from organic acids and bases, such as sodium citrate or sodium ascorbate. The pharmaceutical carrier may also include one or more salts, such as sodium chloride or potassium chloride. The dry powder formulations can be suspended in the liquid formulations to form nanoparticle solutions, and administered systemically or regionally using methods known in the art for the delivery of liquid formulations.

### 2. Liquid Formulations

In some forms, the antiviral peptides are formulated as a liquid. Suitable liquid carriers include, but are not limited to, distilled water, de-ionized water, pure or ultrapure water, saline, and other physiologically acceptable aqueous solutions containing salts and/or buffers, such as phosphate buffered saline (PBS), Ringer's solution, and isotonic sodium chloride, or any other aqueous solution acceptable for administration to an animal or human.

Liquid formulations may include one or more suspending agents, such as cellulose derivatives, sodium alginate, polyvinylpyrrolidone, gum tragacanth, or lecithin. Liquid formulations may also include one or more preservatives, such as ethyl or n-propyl p-hydroxybenzoate.

Formulations may be prepared using one or more pharmaceutically acceptable excipients, including diluents, preservatives, binders, lubricants, disintegrators, swelling agents, fillers, stabilizers, and combinations thereof. Liquid formulations may also contain minor amounts of polymers, surfactants, or other excipients well known to those of the art. In this context, “minor amounts” means no excipients are present that might adversely affect the delivery of the antiviral peptide compositions to organs or tissues, e.g., through circulation.

In some forms, the antiviral peptides are formulated in a suitable carrier. A carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof.

In some forms, the antiviral peptides are formulated to contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s). Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzyl peroxide, benzethonium chloride, benzyl alcohol, cetylpyridinium chloride, chlorobutanol, phenol, phenylethyl alcohol, and thimerosal.

In some forms, the antiviral peptides are formulated to be buffered to a pH, for example, pH 2, 3, 4, 5, 6, 7, 8, 9 or pH 10. In an exemplary form, the formulation is typically buffered to a pH of 3-8 for parenteral administration. Suitable buffers include, but are not limited to, phosphate buffers, acetate buffers, and citrate buffers.

In some forms, the antiviral peptides are formulated to include one or more water soluble polymers. Water soluble polymers are often used in formulations for parenteral administration. Suitable water-soluble polymers include, but are not limited to, polyvinylpyrrolidone, dextran, carboxymethylcellulose, and polyethylene glycol.

Generally, dispersions are prepared by incorporating the various sterilized gelators, stabilizing agents, and/or active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Formulations may be prepared as described in standard references such as “Pharmaceutical dosage form tablets”, eds. Liberman et. al. (New York, Marcel Dekker, Inc., 1989), “Remington-The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment and process for preparing tablets and capsules and delayed release dosage forms of tablets, capsules, and granules.

### 3. Dosage Units

The antiviral peptide compositions are preferably formulated in dosage unit form for ease of administration and uniformity of dosage. The phrase “dosage unit form” refers to a physically discrete unit of conjugate appropriate for the patient to be treated. It will be understood, however, that the total single administration of the compositions will be decided by the attending physician within the scope of sound medical judgment. The therapeutically effective dose can be estimated initially either in cell culture assays or in animal models, usually mice, rats, rabbits, dogs, or pigs. The animal model is also used to achieve a desirable concentration range and route of administration. Such information should then be useful to determine useful doses and routes for administration in humans.

### IV. Methods of Use

Methods for preventing or treating one or more symptoms of coronavirus infection in the subject are described. The methods administer an effective of the antiviral peptides or pharmaceutical formulations thereof to treat or prevent a disease, for example severe acute respiratory syndrome coronavirus 2 (SARS-CoV-2). The antiviral peptides may be administered in any appropriate pharmaceutical carrier, such as a liquid, for example water, and saline, or a powder, for administration to the respiratory system. The formulations can be delivered by any method and/or device which is currently used for pulmonary delivery. For example, nebulizers and inhalers can be used.

Aerosol dosage, formulations and delivery systems may be selected for a particular therapeutic application, as described, for example in Gonda, I. “Aerosols for delivery of therapeutic and diagnostic agents to the respiratory tract,” in Critical Reviews in Therapeutic Drug Carrier Systems, 6:273-313 (1990), and in Moren, “Aerosol dosage forms and formulations,” in Aerosols in Medicine. Principles, Diagnosis and Therapy, Moren, et al., Eds., Esevier, Amsterdam, 1985, the disclosures of which are incorporated herein by reference.

### A. Subjects to be Treated

A subject in need of treatment is a subject having or at risk of having an infection e.g., a subject having or at risk of contracting a viral infection. The methods are particularly suited for those at risk of exposure to one or more respiratory pathogens such as SARS-CoV-2. Thus, in some forms, the subject has not experienced any symptoms from COVID but is at risk of doing so.

A positive SARS-CoV-2 viral test (i.e., reverse transcription polymerase chain reaction [RT-PCR] test or antigen test) or serologic (antibody) test can help assess for current or previous infection. In some forms, the methods retard the development of symptoms in a patient identified as positive via one or more of the SARS-CoV-2 viral tests, with or without any symptoms.

In some forms, the methods provide an effective amount of antiviral peptides to treat or prevent one or more symptoms of coronavirus infection in the subject, for example, reducing or preventing one or more symptoms or physiological markers of severe acquired respiratory syndrome (SARS) in a subject. Exemplary symptoms of COVID-19 include cough, fatigue, fever, body aches, headache, sore throat, loss or altered sense of taste and/or smell, vomiting, diarrhea, cytokine storm, skin changes, ocular complications, confusion, chronic neurological impairment, chest pain and shortness of breath. Therefore, in some forms, the methods prevent or reduce one or more of cough, fatigue, fever, body aches, headache, sore throat, loss or altered sense of taste and/or smell, vomiting, diarrhea, cytokine storm, skin changes, ocular complications, confusion, chronic neurological impairment, chest pain and shortness of breath.

In some forms, the methods reduce or prevent infection by the causative viral disease COVID-19 in a subject. In other forms, the methods prevent or reduces the invading viral pathogens in getting inside and/or proliferating in one or more targeting cells.

In some forms, the coronavirus is a variant of SARS-CoV-2, such as SARS-CoV-2 B.1.1.7 (Alpha variant), SARS-CoV-2 B.1.351 (Beta variant), SARS-CoV-2 P.1 (Gamma variant), SARS-CoV-2 B.1.617, SARS-CoV-2 B.1.617.1 (Kappa variant), SARS-CoV-2 B.1.621 (Mu variant), SARS-CoV-2 B.1.617.2 (Delta variant), SARS-CoV-2 B.1.617.3, and SARS-CoV-2 B.1.1.529 (Omicron variant).

### B. Methods of Administration

The compositions are generally administered to a subject in an effective amount. As used herein the term “effective amount” means a dosage sufficient to inhibit, or prevent one or more infections, or symptoms of a disease or to otherwise provide a desired pharmacologic and/or physiologic effect. The precise dosage will vary according to a variety of factors such as subject-dependent variables (e.g., age, immune system health, etc.), the specific variant of virus, and the treatment being affected.

The pharmaceutical compositions can be for administration by parenteral (intramuscular, intraperitoneal, intravenous, or subcutaneous injection), transdermal (either passively or using iontophoresis or electroporation), or transmucosal (nasal, vaginal, rectal, or sublingual) routes of administration or using bioerodible inserts and can be formulated in dosage forms appropriate for each route of administration.

In preferred forms, the compositions are administered locally, for example by intranasal administration. Typically, local administration causes an increased localized concentration of the compositions which is greater than that which can be achieved by systemic administration. In some forms, the compositions are delivered locally to the appropriate cells by using a catheter or syringe. Other means of delivering such compositions locally to cells include using infusion pumps (for example, from Alza Corporation, Palo Alto, Calif.) or incorporating the compositions into polymeric implants (see, for example, P. Johnson and J. G. Lloyd-Jones, eds., Drug Delivery Systems (Chichester, England: Ellis Horwood Ltd., 1987), which can affect a sustained release of the particles to the immediate area of the implant.

In one form, the method includes administration via a nebulizer to a subject of an effective amount of a composition containing the antiviral peptides.

### 1. Route of Administration

The antiviral peptides are particularly suited for administration to the nasal or pulmonary system or administered to a mucosal surface. The compositions may be administered as a dry powder, as an aqueous suspension (in water, saline, buffered saline, etc), in a hydrogel, or liposome, in capsules, tablets, troches, or other standard pharmaceutical excipient.

In some forms, the antiviral peptides or formulations thereof are administered via a nebulizer. A nebulized solution is one dispersed in air to form an aerosol, and a nebulizer generates very fine liquid droplets suitable for inhalation into the lung. Nebulizers typically use compressed air, ultrasonic waves, or a vibrating mesh to create a mist of the droplets and may also have a baffle to remove larger droplets from the mist by impaction. A variety of nebulizers are available for this purpose, such as ultrasonic nebulizers, jet nebulizers and breath-actuated nebulizers.

The deposition of inhaled medication into the lung and airways is influenced by multiple factors, including the characteristics of the nebulizer device, the formulation properties of the aerosol, the patient's breathing pattern, airway geometry, and potential differences in regional airway ventilation. Differences among nebulizer systems can impact by several-fold the efficiency of drug delivery to the lung. Nebulizers are therefore selected to efficiently deliver the desired amount of formulations to the targeted areas of the lung, with minimized drug-related adverse effects.

One important consideration in choosing an appropriate nebulizer is its ability to generate appropriate droplet size of the aerosol for pulmonary treatment. Generally, small droplet sizes are preferred for more efficient delivery of the formulations to reach the lower respiratory tract. The amount of drug in small droplets (<5 μm), commonly described as the fine-droplet fraction, is the portion of an aerosolized drug most efficiently delivered to the distal airways. In some forms, droplet size of less than 5.0 μm is suitable, for example about 1.5 μm-5.0 μm, preferably droplet size of about 2.5 μm-3.5 μm or less.

In some forms, the antiviral peptides or formulations thereof are administered via an inhaler. Inhalers are small, handheld devices that deliver a puff of medicine into the airways. There are three basic types: metered-dose inhalers (MDIs), dry powder inhalers (DPIs), and soft mist inhalers (SMI).

### C. Treatment Regimens

A treatment regimen can include one or multiple administrations of the antiviral peptides and formulations thereof for achieving a desired physiological change, including administering to an animal, such as a mammal, especially a human being, an effective amount of the compositions to treat the disease or symptom thereof, or to produce the physiological change. In preferred forms, the desired physiological change is the reduction in the amount of syncytial formation and lung damage in the subject.

### 1. Dosage and Effective Amounts

A therapeutically effective amount of antiviral peptides used in the treatment of diseases and disorders associated with coronavirus infection are typically sufficient to reduce or alleviate one or more symptoms of the diseases and disorders associated with coronavirus infection. Symptoms of diseases and disorders associated with coronavirus infection may be cough, fatigue, fever, body aches, headache, sore throat, loss or altered sense of taste and/or smell, vomiting, diarrhea, cytokine storm, skin changes, ocular complications, confusion, chronic neurological impairment, chest pain and shortness of breath. Accordingly, the amount of antiviral peptides can be effective to, for example, treat or prevent one or more symptoms of a coronavirus infection. Preferably the antiviral peptides are delivered topically to the mucosal surface of the lung. Preferably the antiviral peptides do not target or otherwise modulate other metabolic processes or metabolic products. In some forms, the antiviral peptides are administered in an effective amount to coronavirus infection, or one or more diseases or disorders associated with coronavirus infection in a subject at risk of exposure to SAR-Cov-2 virus.

The actual effective amounts of antiviral peptides can vary according to factors including the specific antiviral peptides administered, the particular composition formulated, the mode of administration, and the age, weight, condition of the subject being treated, as well as the route of administration and the disease or disorder.

In some forms, the effective amount of antiviral peptides causes little or no killing of cells within the subject, and preferably little or no inhibition of metabolism in cells. It is particularly preferred that the composition does not dampen activities of immune cells.

In some forms, dosages of antiviral peptides are administered once, twice, or three times daily, or every other day, two days, three days, four days, five days, or six days to a human. In some forms, dosages of antiviral peptides are administered about once or twice every week, every two weeks, every three weeks, or every four weeks. In some forms, dosages are administered about once or twice every month, every two months, every three months, every four months, every five months, or every six months.

In some forms, the regimen includes one or more cycles of a round of therapy with antiviral peptides followed by a drug holiday (e.g., no antiviral peptides). The round of the therapy can be, for example, any of the administrations discussed above. Likewise, the drug holiday can be 1, 2, 3, 4, 5, 6, or 7 days; or 1, 2, 3, 4 weeks, or 1, 2, 3, 4, 5, or 6 months.

In particular forms, the subject is administered a dosage of between about 0.1 mg/kg body weight and 100 mg/kg body weight, inclusive, of antiviral peptides. In some forms, the subject is administered a dosage of between about 0.1 mg/kg body weight and 10 mg/kg body weight, inclusive, of antiviral peptides. In some forms, the subject is administered a dosage of between about 0.2 mg/kg body weight and 10 mg/kg body weight, inclusive, of antiviral peptides. In some forms, the subject is administered a dosage of between about 0.3 mg/kg body weight and 10 mg/kg body weight, inclusive, of antiviral peptides. In some forms, the subject is administered a dosage of between about 0.4 mg/kg body weight and 10 mg/kg body weight, inclusive, of antiviral peptides. In some forms, the subject is administered a dosage of between about 0.5 mg/kg body weight and 10 mg/kg body weight, inclusive, of antiviral peptides. In some forms, the subject is administered a dosage of between about 1.0 mg/kg body weight and 10 mg/kg body weight, inclusive, of antiviral peptides. In some forms, the subject is administered a dosage of between about 1.0 mg/kg body weight and 5 mg/kg body weight, inclusive, of antiviral peptides. Particular dosage regimens include, for example, one or more cycles in which the subject is administered the antiviral peptides on each of two, three, four, five, six or seven days, weeks or months in a row, followed by a one, two, three, four, five, six or seven-day, week, or month drug holiday.

In the most preferred forms, methods of using the antiviral peptides lead to direct or indirect reduction in the syncytial formation and lung damage, increase in the quality of life of those suffering from the disease, decrease in the dose of other medications required to treat the disease, delaying the progression of the disease, and/or prolonging survival of individuals.

### 2. Controls

The effect of the antiviral peptide compositions can be compared to a control. Suitable controls are known in the art and include, for example, an untreated subject, or a placebo-treated subject. A typical control is a comparison of a condition or symptom of a subject prior to and after administration of the targeted agent. The condition or symptom can be a biochemical, molecular, physiological, or pathological readout. For example, the effect of the composition on a particular symptom, pharmacologic, or physiologic indicator can be compared to an untreated subject, or the condition of the subject prior to treatment. In some forms, the symptom, pharmacologic, or physiologic indicator is measured in a subject prior to treatment, and again one or more times after treatment is initiated. In some forms, the control is a reference level, or average determined based on measuring the symptom, pharmacologic, or physiologic indicator in one or more subjects that do not have the disease or condition to be treated (e.g., healthy subjects). In some embodiments, the effect of the treatment is compared to a conventional treatment that is known the art.

### D. Combination Therapies and Procedures

The antiviral peptide compositions can be administered alone or in combination with one or more conventional therapies. In some forms, the conventional therapy includes administration of one or more of the compositions in combination with one or more additional active agents. The combination therapies can include administration of the active agents together in the same admixture, or in separate admixtures. Therefore, in some forms, the pharmaceutical composition includes two, three, or more active agents. Such formulations typically include an effective amount of an agent targeting the site of treatment. The additional active agent(s) can have the same or different mechanisms of action. In some forms, the combination results in an additive effect on the treatment of the lung condition. In some forms, the combinations result in a more than additive effect on the treatment of the disease or disorder.

The additional therapy or procedure can be simultaneous or sequential with the administration of the dendrimer composition. In some forms, the additional therapy is performed between drug cycles or during a drug holiday that is part of the dosage regime. For example, in some forms, the additional therapy or procedure is damage control surgery, fluid resuscitation, blood transfusion, bronchoscopy, and/or drainage.

In some forms, the antiviral peptide formulation is used in combination with oxygen therapy. In further forms, the additional therapy or procedure is prone positioning, recruitment maneuver, inhalation of NO, extracorporeal membrane oxygenation (ECMO), intubation, and/or inhalation of PGI₂. A prone position enhances lung recruitment in a potentially recruitable lung by various mechanisms, releasing the diaphragm, decreasing the effect of heart and lung weight and shape on lung tissue, decreasing the lung compression by the abdomen, and releasing the lower lobes, which improves gas exchange and decreases mortality in severe ARDS patients. ECMO provides extracorporeal gas exchange with no effect on lung recruitment. It affords lung rest and works well for the non-recruitable lung. It has been shown to improve survival for certain groups of patients in high-performance ECMO centers. Additional therapeutic agents can also include one or more of antibiotics, surfactant, corticosteroids, and glucocorticoids.

In some forms, the compositions and methods are used prior to or in conjunction, subsequent to, or in alternation with treatment with one or more additional therapies or procedures.

### V. Kits

Kits are also disclosed. The kit can include a single dose or a plurality of doses of a composition including one or more of the antiviral peptides, or pharmaceutical formulation thereof, and instructions for administering the compositions. Specifically, the instructions direct that an effective amount of the antiviral peptidecomposition be administered to an individual at risk of exposure to one or more respiratory pathogens such as severe acute respiratory syndrome (SARS) virus. The composition can be formulated as described above with reference to a particular treatment method and can be packaged in any convenient manner.

The present invention will be further understood by reference to the following non-limiting examples.

### EXAMPLES

### Example 1: Identification of a Human Peptide 4H30 Derived from Human Beta Defensin 2 (HBD2) for Inhibiting SARS-CoV-2 Variants, H1N1 Virus, HRV-1B and Major Group HRV-B14 Viruses

### Material and Methods

### Cell and Virus Cultures

Madin Darby canine kidney (MDCK, CCL-34), 293T (CRL-3216), A549 (CCL-185), VeroE6 (CRL-1586), VeroE6-TMPRSS2 (VeroE6-T), Calu-3 (HTB-55) (H. Zhao, et al., Nat Commun, 12 (2021) 1517) cells from ATCC were cultured in Dulbecco minimal essential medium (DMEM) or DMEM-F12K supplemented with 10% fetal bovine serum (FBS), 100 IU ml⁻¹ penicillin and 100 μg ml⁻¹ streptomycin. The virus strains used in this study included SARS-CoV-2 variants (H. Zhao, et al., Nat Commun, 11 (2020) 4252; and L.L. Chen, et al., Clin Infect Dis, (2021)). SARS-CoV-2 variants were cultured in VeroE6 or VeroE6-T cells and viral titers were determined by plaque assay.

### Synthesis of Peptides

H23, H26, H30, 2H30, and 4H30 shown in Table 1 were synthesized by ChinaPeptide (Shanghai, China). All peptides were dissolved in water. The solubility of peptide in water is greater than 5 mg ml⁻¹. The purity of all peptides was >80%. The purity and mass of each peptide were verified by HPLC and mass spectrometry.

### Plaque Reduction Assay

Antiviral activity of peptides was measured using a plaque reduction assay (H. Zhao, et al., Nat Commun, 9 (2018) 2358). Briefly, peptides were dissolved in PBS or 30mM phosphate buffer (PBS/5) containing 24.6 mM Na₂HPO₄ and 5.6 mM KH₂PO₄ at a pH of 7.4. Peptides or bovine serum albumin (BSA, 0.2-25.0 μg ml⁻¹) were premixed with SARS-CoV-2 at room temperature. After 45-60 min of incubation, peptide-virus mixture was transferred to Vero-E6 cells, correspondingly. At 1 h post infection, infectious media were removed, and 1% low melting agar was added to cells. Cells were fixed using 4% formalin at 2-3 day post infection. Crystal blue (0.1%) was added for staining, and the number of plaques was counted.

### Cytotoxicity Assay

Cytotoxicity of peptides was determined by the detection of 50% cytotoxic concentration (CC₅₀) using a tetrazolium-based colorimetric MTT assay (H. Zhao, et al., Sci Rep, 6 (2016) 22008). Briefly, cells were seeded in 96-well cell culture plate at an initial density of 4×10⁴ cells per well in DMEM supplemented with 10% FBS and incubated for overnight. Cell culture media were removed and then DMEM supplemented with various concentrations of peptides and 1% FBS were added to each well. After 24 h incubation at 37° C., MTT solution (5 mg ml⁻¹, 10 μl per well) was added to each well for incubation at 37° C. for 4 h. Then, 100 μl of 10% SDS in 0.01M HCl was added to each well. After further incubation at room temperature with shaking overnight, the plates were read at OD570 using VictorTM X3 Multilabel Reader (PerkinElmer, USA). Cell culture wells without peptides were used as the experiment control and medium only served as a blank control.

### Transmission Electron Microscopy Assay

To determine the effect of 4H30 on viral particles, SARS-CoV-2 was pretreated by 100 μg ml⁻¹ of 4H30 or H30 for 1 h. The virus was fixed by formalin for overnight. For spike treated by 4H30 or H30, spike (100 μg ml⁻¹ mixed with 50 μg ml⁻¹ of 4H30 or H30. Viral or protein samples were applied to continuous carbon grids. The grids were transferred into 4% uranyl acetate and incubated for 1 min. After removing the solution, the grids were air-dried at room temperature. For each peptide/DNA nanoparticle, three independent experiments were done for taking TEM images by FEI Tecnal G2-20 TEM or Philips CM100-TEM.

### ELISA Assay

For ELISA assay (H. Zhao, et al., Nat Commun, 9 (2018) 2358), peptides (1.0 μg per well) dissolved in H₂O or GAGs (CS or HS) dissolved in PBS were coated onto ELISA plates and incubated at 4° C. overnight and was blocked at 4° C. overnight. For determining spike and ACE2 binding, 60 ng S or ACE2 in PBST was incubated with peptides at 37° C. for 30 min. The binding abilities of S or ACE2 proteins to peptides were determined by incubation with rabbit anti-spike (Sino, Cat^{#} 40590-T62, 1: 8,000) or rabbit anti-ACE2 (Takara, Cat^{#} A4612, 1:6000) at 37° C. for 30 min and then incubation with goat-anti-rabbit HPR (Life Technologies, Cat^{#} 656120, 1:4000) at 37° C. for 30 min. The reaction was developed by adding 100 μl of TMB single solution (Life Technologies, Cat^{#} 002023) for 15 min at 37° C. and stopped with 50 μl of 1 M H₂SO₄. Readings were obtained in an ELISA plate reader (Victor 1420 Multilabel Counter; PerkinElmer) at 450 nm.

### Viral RNA Extraction and RT-qPCR

Viral RNA was extracted by Viral RNA Mini Kit (QIAGEN, Cat^{#} 52906, USA) according to the manufacturer's instructions. Real-time RT-qPCR was performed as we described previously (H. Zhao, et al., Nat Commun, 9 (2018) 2358). Extracted RNA was reverse transcribed to cDNA using PrimeScript II 1^{st} Strand cDNA synthesis Kit (Takara, Cat^{#} 6210A) using GENEAMP® PCR system 9700 (Applied Biosystems, USA). The cDNA was then amplified using specific primers (Table 2) for detecting SARS-CoV-2using LIGHTCYCLE® 480 SYBR Green I Master (Roach, USA). For quantitation, 10-fold serial dilutions of standard plasmid equivalent to 10¹ to 10⁶ copies per reaction were prepared to generate the calibration curve. Real-time qPCR experiments were performed using LIGHTCYCLER® 96 system (Roche, USA).

TABLE 2
Forward and reverse primer sequences for SARS-CoV-2

| Gene | Primer | Oligonucleotide sequence (5′ to3′) |
|---|---|---|
| SARS-CoV-2 | S-F | CCTACTAAATTAAATGATCTCTGCTTTACT (SEQ |
| | | ID NO: 5) |
| | S-R | CAAGCTATAACGCAGCCTGTA (SEQ ID NO: 6) |

### Endosomal Acidification Assay

Endosomal acidification was detected with a pH-sensitive dye (pHrodo green dextran, Invitrogen, Cat^{#} P35365) according to the manufacturer's instructions as previously described but with slight modification (H. Zhao, et al., Nat Commun, 9 (2018) 2358). First, VeroE6 cells were treated with DMEM, 4H30 (25.0 μg ml⁻¹), or bafilomycin A1 (50.0 nM) at 4° C. for 15 min. Second, VeroE6 cells were added with 100 μg ml⁻¹ of pH-sensitive dye and DAPI and then incubated at 4° C. for 15 min. Before taking images, cells were further incubated at 37° C. for 15 min and then cells were washed twice with PBS. Finally, PBS was added to cells and images were taken immediately with confocal microscope (Carl Zeiss LSM 800, Germany).

### Nucleoprotein (NP) Immunofluorescence Assay

According to previous experiments (H. Zhao, et al., Nat Commun, 9 (2018) 2358), VeroE6 cells were seeded on cell culture slides and were infected with SARS-CoV2 at 0.01 MOI pretreated with DMEM or 4H30 (50.0 μg ml⁻¹). After 18 h post infection, cells were fixed with 4% formalin for 1 h and then permeabilized with 0.2% Triton X-100 in PBS for 5 min. Cells were washed by PBS and then blocked by 5% BSA at room temperature for 1 h. Cells were incubated with rabbit IgG anti-NP (M. L. Yeung, et al., Cell, 184 (2021) 2212-2228.e2212) (1:4000) at room temperature for 1 h and then washed by PBS for next incubation with goat anti-rabbit IgG Alexa-488 (Life Technologies, Cat^{#} A32731, 1:600) at room temperature for 1 h. Finally, cells were washed by PBS and stained with DAPI. Images were taken by confocal microscope (Carl Zeiss LSM 800, Germany).

### Viral Entry Fluorescence Assay

To identify the effect of 4H30 on virus, SARS-CoV-2 was pre-labelled by green Dio dye (Invitrogen, Cat^{#} 3898) according to the manufacture introduction. Dio-labeled virus was treated by DMEM, 4H30 or H30 (25 μg ml⁻¹) for 45 min. VeroE6 or Calu-3 cells were infected by the pre-treated virus for 1 h. Virus and cells were fixed by 4% formalin. Cell membrane was stained by membrane dye Alexa 594 (red, Invitrogen, W11262) and cell nucleus were stained by DAPI (blue). Virus entry or without entry on cell membrane was determined by confocal microscope (Carl Zeiss LSM 800, Germany).

### Viral Release Assay

SARS-CoV-2 (0.01 MOI) was used to infect VeroE6 cells. At 6 h or 14 h post-infection, viral culture supernatants were removed. DEME with or without 4H30 (50.0 μg ml⁻¹) was added to infected cells. At 10 h or 18 h post-infection, supernatants were collected to measure viral titers by RT-qPCR or fixed for anti-Spike immunofluorescence assay. After fixing at room temperature for 45 min, cells were blocked by 5% BSA for 1 h. Rabbit-anti-spike (Sino, Cat #40590-T62, 1:6,000) and goat anti-rabbit IgG Alexa-488 (Life Technologies, Cat^{#} A32731, 1:600) at room temperature for 45 min. Images were taken by confocal microscope (Carl Zeiss LSM 800, Germany).

### Spike-ACE2 Mediated Cell Fusion Assay

According to previous study (S. Xia, et al., Signal Transduct Target Ther, 5 (2020) 92), the pSpike of SARS-CoV-2, pACE2-human, or pGFP were transfected to 293T cells for protein expression. After 24 hours, to trigger the spike-ACE2 mediated cell fusion, 293T-Spike-GFP cell were co-cultured with 293T-ACE2 or Calu-3 cells with the supplement of drugs. The 293T-GFP cells were co-cultured with 293T-ACE2 or Calu-3 cells as the negative control. After 6-8 h of co-culture, five fields were randomly selected in each well to take the cell fusion pictures by fluorescence microscopes.

### Western Blot

Spike, ACE2 (7.5 μg) or PBS was premixed with 4H30 (10 μg) and then was passed through 30 kDa centrifugal filters to elute the unbinding 4H30. Collected the remained proteins in the centrifugal filters and loaded the samples to 15% PAGE to do western blot. 4H30 was detected by anti-HBD2, (Thermo Scientific, Cat #PA5-103126, 1:5000) and goat anti-rabbit HRP as secondary antibody (Thermo Scientific, 656120, 1:4000 dilution).

### Antiviral Assay in Animals

Female hamsters (6-8 week old) (J. F. Chan, et al., Clin Infect Dis, (2020)) were kept in biosafety level 2/3 laboratory (housing temperature between 22˜25° C. with dark/light cycle) and given access to standard pellet feed and water ad libitum. All experimental protocols followed the standard operating procedures of the approved biosafety level 2/3 animal facilities. Animal ethical regulations were approved by the Committee on the Use of Live Animals in Teaching and Research of the University of Hong Kong (B. J. Zheng, et al., Proc Natl Acad Sci U S A, 105 (2008) 8091-8096). To evaluate the drug toxicity in vivo, hamsters and mice were intranasally inoculated with 4H30 (0.5 mg kg⁻¹) was intranasally inoculated to hamster lungs before or after viral challenge. Two more doses were given to mice in the following day. Lung tissues were harvested at day 4 for H&E staining. To evaluate the pre-exposure antiviral activity, 4H30, H23 or PBS was intranasally inoculated to hamster lungs at 24 h, 8 h, 4 h, 0.05 h (Pre) before SARS-CoV-2 inoculation. Lung tissues were collected at day 2 post-infection for determining viral titers and histopathology. To evaluate the post-exposure antiviral activity, hamsters were intranasally inoculated with SARS-CoV-2 (B.1.1.63 or B.1.617.2) to lungs. At 8 h post infection, PBS, were given to animals. Two more doses were given to mice/hamsters in the following day. Viral loads in mouse/hamster lungs were measured at day 2 post infection by plaque assay.

### Results

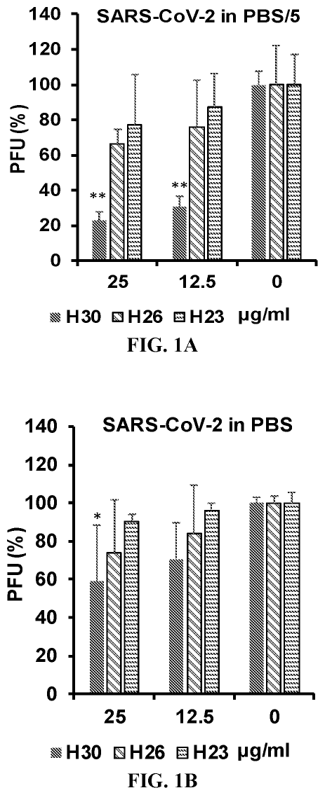

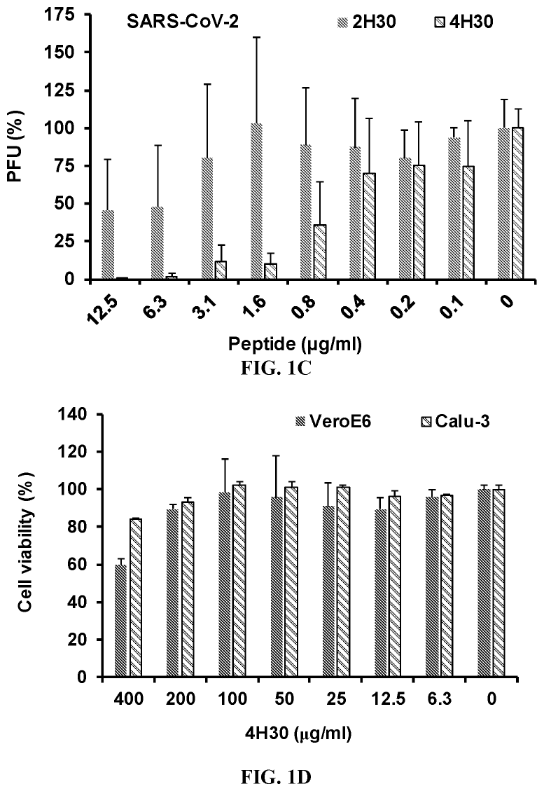

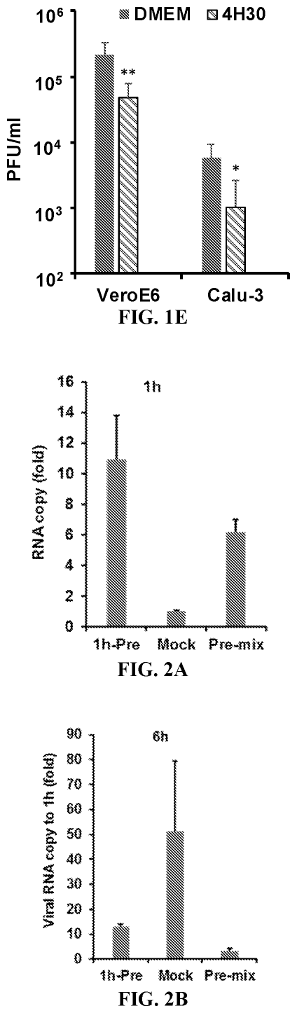

Human beta defensin 2 (HBD2) was demonstrated to have antimicrobial activity (M. K. Holly, et al., Annu Rev Virol, 4 (2017) 369-391). However, defensin peptides generally showed decreased antimicrobial activity in physiological salt condition when compared with their activity in low salt condition (H. Zhao, et al., Nat Commun, 12 (2021) 1517; R. Bals, et al., J Clin Invest, 102 (1998) 874-880). Decreased antimicrobial activity of defensin peptides in physiological salt condition poses a barrier for developing them as antimicrobials in vivo despite their broad-spectrum antimicrobial activities in vitro. To find a human-sourced antiviral peptide with effective antiviral activity in physiological salt condition, a 30-amino acid peptide (H30) derived from HBD2 was first identified which inhibited SARS-CoV-2 in PBS but more effective in low salt condition (FIGS. 1A and 1B). H23, H26, H30, 2H30, and 4H30 shown in Table 1 were all synthesized. The antiviral activity of these peptides against SARS-CoV-2 in low salt condition (30 mM phosphate buffer, PBS/5) and high salt condition (150 mM PBS) were determined by plaque reduction assays (FIGS. 1A and 1B). The weak antimicrobial activity of H30 in PBS was consistent with the decreased antimicrobial activity of HBD2 and other defensins in physiological salt condition (R. Bals, et al., J Clin Invest, 102 (1998) 874-880; E. Guaní-Guerra, et al., Clin Immunol, 135 (2010) 1-11; C. Xu, et al., Viruses, 13 (2021)).

Next, two branched H30 (2H30) and four branched H30 (4H30) were synthesized. The antiviral assays indicated that 4H30 significantly inhibited SARS-CoV-2 infection (IC₅₀=0.59 μg ml⁻¹) which is more potent than H30 (IC₅₀>25 μg ml⁻¹) and 2H30 (FIG. 1C) in PBS. The cytotoxicity assay indicated that the TC50 of 4H30 in VeroE6 and Calu-3 cells was higher than 400 μg ml⁻¹ (FIG. 1D). To confirm the antiviral activity of 4H30, the anti-NP staining result confirmed that viral replication was significantly inhibited and restricted in very few cells when compared with untreated virus (data not shown). Furthermore, it was demonstrated that 4H30 significantly inhibited SARS-CoV-2 replication in VeroE6 and Calu-3 cells when 4H30 was added to cells at 6 hours post-infection (hpi) (FIG. 1E). These results indicated that branched 4H30 potently inhibit SARS-CoV-2 replication when treated by 4H30 before or after virus challenge in physiological salt condition.

### 4H30 Blocks SARS-CoV-2 Entry

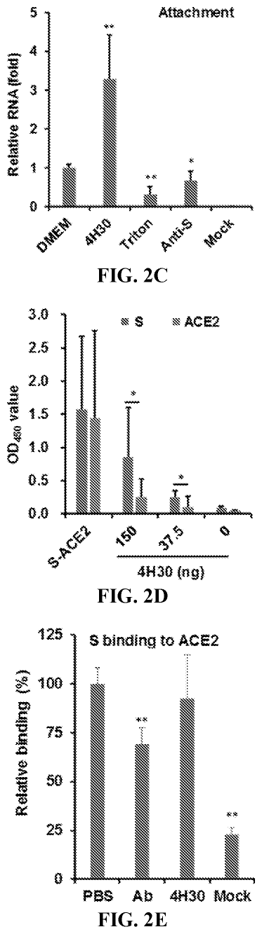

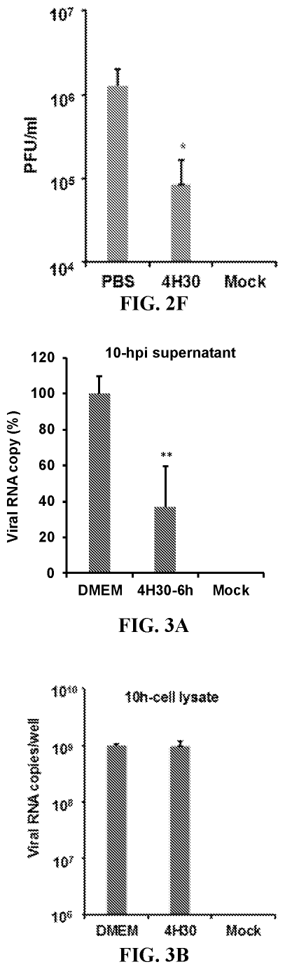

Next, the antiviral mechanism of 4H30 was investigated. Viral attachment and/or entry to VeroE6 cells was significantly increased at 1 hour post-infection (hpi) when cells were pretreated by 4H30 or when virus was pretreated by 4H30 (FIG. 2A). At 6 hpi, virus replication in cell lysate was significantly inhibited when virus was pretreated by 4H30 as demonstrated by no significant increase in viral load when compared with viral load at 1 hpi (FIG. 2B), which was consistent with plaque reduction assay when virus was pretreated by 4H30 (FIG. 1C). However, PBS-treated virus showed 50-fold viral load increase at 6 hpi when compared with viral load at 1 hpi. Compared with viral load at 1 hpi, there was 13-fold viral load increase at 6 hpi when cells were pretreated by 4H30 before virus challenge, which indicated that the increased virus attachment and/or entry at 1 hpi did not increase the viral replication when compared with PBS-treated virus (FIG. 2B).

Next, 4H30 was also shown to increase SARS-CoV-2 attachment to cells at 4 degrees (FIG. 2C), which was consistent with the increased viral load at 1 hpi (FIG. 2A). Furthermore, 4H30 more effectively bound to spike when compared with ACE2 (FIG. 2D), while 4H30 did not significantly affect the binding between spike and ACE2 (FIG. 2E). These results indicated that 4H30 did not directly cross-link spike with ACE2 to improve viral attachment because 4H30 was able to bind to spike but not effectively to ACE2. Moreover, when 1×10⁶ PFU ml⁻¹ of SARS-CoV-2 was pretreated by 50 μg ml⁻¹ of 4H30 and then the 4H30-treated virus was 10,000-fold diluted for plaque assay, 4H30 (at 0.005 μg ml⁻¹<IC₅₀=0.59 μg ml⁻¹) significantly inhibited SARS-CoV-2 infection (FIG. 2F), which indicated that 4H30 significantly inhibited viral infection by irreversible binding to virus before viral entry. Collectively, these results indicated that 4H30 increased viral attachment to cells but still effectively inhibited viral replication following viral entry.

Next, 4H30 was shown to cross-link SARS-CoV-2 to form big viral clusters on Calu-3 and VeroE6 cell membrane without viral entry using fluorescent microscopy, which indicated that cross-linked viral particles did not infect cells through the two entry pathways of SARS-CoV-2 mediated by surface membrane fusion in Calu-3 cells with TMPRSS2⁺ or by endocytosis in VeroE6 cells. To further confirm that 4H30 could cluster viral particles, it was further demonstrated that 4H30 cross-linked SARS-CoV-2 to form bigger viral clusters under transmission electron microscopy (TEM). The intact viral particles under TEM indicated that 4H30 did not disrupt viral particles. Because 4H30 was able to bind to spike (FIG. 2D), it is conceivable that 4H30 could also cross-link spike protein as shown under TEM. When Calu-3 cells or VeroE6 cells were treated by 4H30 before virus challenge (4H30-Pre), 4H30 did not cluster viral particles and did not block viral entry, which indicated that 4H30 did not affect host factors such as ACE2 on cell membrane to inhibit virus attachment/entry. Collectively, these results indicated that 4H30 cross-linked SARS-CoV-2 through its binding to spike to form big viral particles and therefore block viral entry through both pathways.

### 4H30 Inhibits SARS-CoV-2 Release

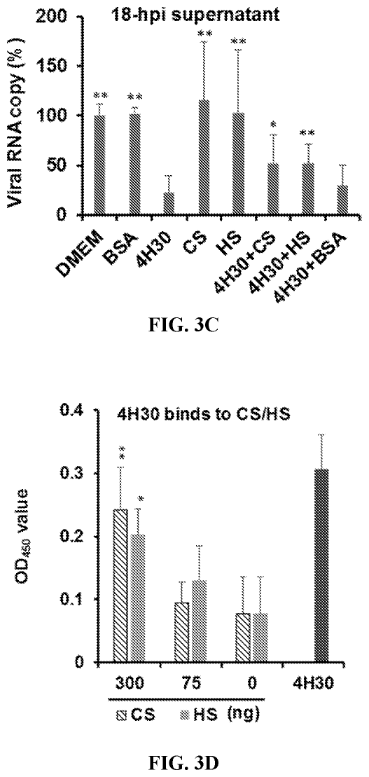

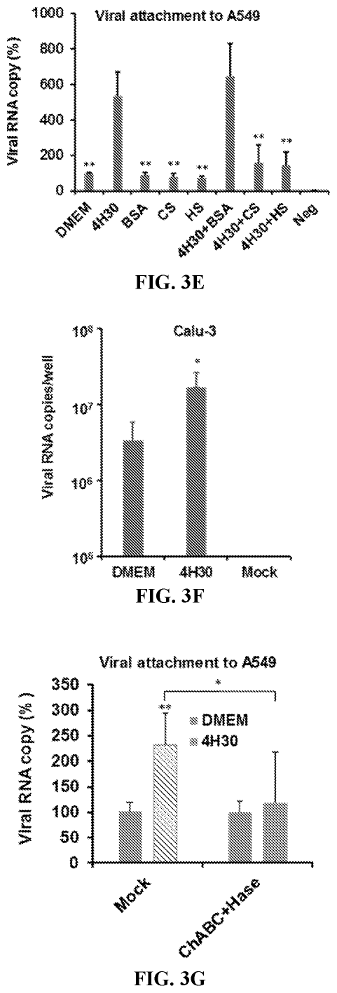

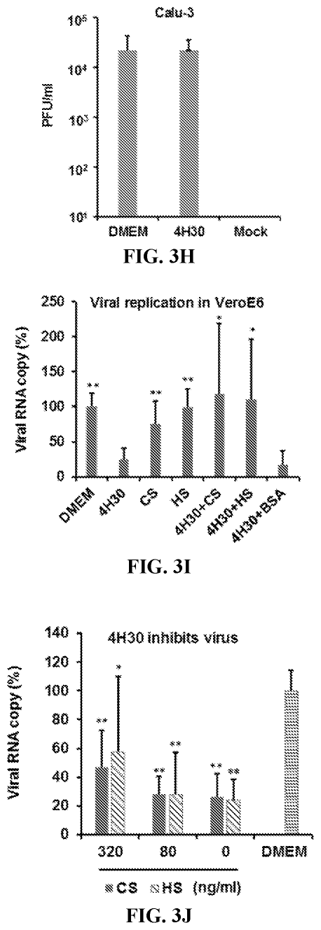

Notably, 4H30 was shown to significantly inhibit viral release as indicated by the reduced viral load of SARS-CoV-2 in cell culture supernatants at 10 hpi when 4H30 was added to infected cells at 6 hpi (FIG. 3A) and the inhibitory effect of 4H30 was not on the intracellular replicative process (FIG. 3B). To confirm that the inhibition of 4H30 was on the step of virus release, immunofluorescence imaging was used. Virus particles were stuck on cell membrane when 4H30 was added to infected cells at 14 hpi with the infected cells were fixed at 18 hpi. The significant decrease in viral load in supernatants at 18 hpi further confirmed that 4H30 significantly inhibited SARS-CoV-2 release when compared with a control (FIG. 3C).

Previous studies showed that HBD2 could bind to glycosaminoglycans (GAGs) (E. S. Seo, et al., Biochemistry, 49 (2010) 10486-10495). It has been shown here that 4H30 could effectively bind to GAGs including chondroitin sulfate (CS) and heparan sulfate (HS) when compared with BSA (FIG. 3D). Moreover, it has been shown that the antiviral activity of 4H30 on viral release was significantly reduced when 4H30 was pretreated by CS or HS, but BSA did not significantly affect the inhibitory activity of 4H30 on SARS-CoV-2 release (FIG. 3C). These results indicated that 4H30 cross-linked spike of SARS-CoV-2 with GAGs to inhibit viral release. To further confirm that 4H30 could cross-link virus with cellular GAGs, it was demonstrated that the attachment of SARS-CoV-2 to cells treated by 4H30 was significantly increased in A549 (ACE2⁻) and Calu-3 (ACE2⁺) cells when compared with untreated cells (FIGS. 3E and 3F), The attachment was significantly reduced when 4H30 was pretreated by GAGs (FIG. 3E) or when cells were pretreated by Chondroitinase ABC (ChABC) and Heparinase (Hase) which could remove cell surface GAGs (FIG. 3G). These results further confirmed that 4H30 cross-links virus with GAGs to increase viral attachment to cells even in the absence of ACE2 in A549 cells (M. L. Yeung, et al., Cell, 184 (2021) 2212-2228.e2212). But the increased virus attachment did not increase SARS-CoV-2 replication in Calu-3 cells (FIG. 3H). This was consistent with the viral replication results in VeroE6 cells when cells were pre-treated by 4H30 (FIG. 2B).

It was further demonstrated that the antiviral activity of 4H30 treated by 1250 ng/ml of CS or HS (FIG. 31) was significantly reduced when compared with untreated 4H30 or BSA-treated 4H30. However, when CS or HS was in the physiological concentration (50˜100 ng/ml) (E. Papakonstantinou, et al., Br J Pharmacol, 157 (2009) 1111-1127), GAG-treated 4H30 could still significantly inhibit viral replication (FIG. 3J), which was consistent with the antiviral activity of 4H30 when it was added to cells after virus challenge. Together with its inhibition on viral entry, these results showed that 4H30could cross-link viral particles and cross-link viral spike with GAGs to achieve the desired antiviral effect through the dual functionality of blocking both steps of virus entry and virus release.

### 4H30 Against SARS-CoV-2 Variants

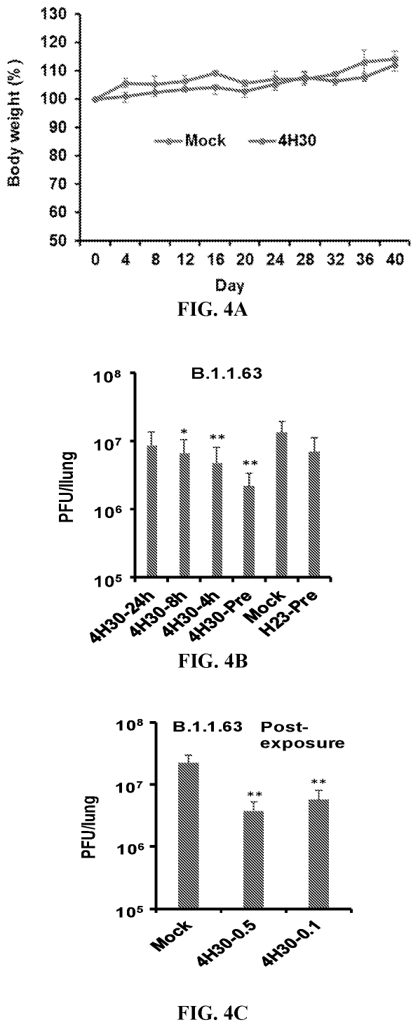

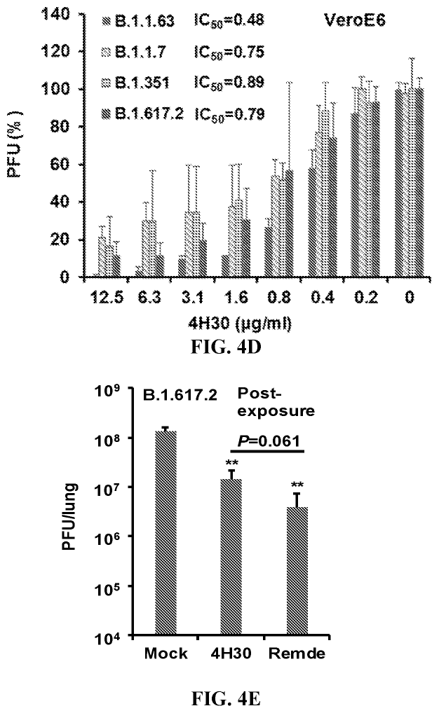

To test for potential toxicity of 4H30 in vivo, pathological and body weight changes after 4H30 was intranasally inoculated into hamsters/mice were monitored. Inhaled 4H30 did not cause pathological and body weight changes at 4 days-post-inoculation (dpi) (FIG. 4A). To determine if 4H30 could cause any lung damage over a longer period, the lung histopathology and body weight change at 40 dpi were monitored. There were no significant histopathological and body weight changes when compared with mock group.

Next, in vivo prophylactic activity of 4H30 against SARS-CoV-2 (B.1.1.63) was tested. It was shown that 4H30 (0.5 mg kg⁻¹) significantly inhibited SARS-CoV-2 replication when one dose of 4H30 was given to hamster lungs at the time (4H30-Pre) before virus challenge. 4H30 significantly inhibited viral replication when one dose of 4H30 was given to hamsters at 4 h before virus challenge (FIG. 4B). 4H30 reduced viral replication by 2.3-fold when given to hamsters at 8 h before virus challenge but did not show antiviral activity when given at 24 h before virus challenge when compared with untreated controls. In addition, syncytia and epithelial cell disorder in bronchial tree were observed in the mock-treated hamster lungs, but these histopathological changes were not found in hamsters treated by 4H30-0.05, which is likely due to the blocking of spike-ACE2 mediated fusion by 4H30. This fusion inhibition by 4H30 could be attributed to the inhibition of endosomal acidification, similar to the activity of bafilomycin Al inhibiting endosomal acidification and cell fusion. Moreover, it was shown that 4H30 did not inhibit the fusion mediated by spike-Calu-3 cells, in which spike-ACE2 mediated fusion relied on TMPRSS2 pathway but not endocytosis pathway. Collectively, these results indicated that, the inhibition of 4H30 on spike-ACE2 mediated fusion could contribute to the reduced pathological changes apart from the inhibitory effect of 4H30 on viral entry and release.

To investigate the post-challenge antiviral activity of 4H30 in vivo, hamsters were challenged with SARS-CoV-2 (B.1.163) and then treated hamsters by intranasal inhalation of 4H30 (0.5 mg kg⁻¹), 4H30 (0.1 mg kg⁻¹) and PBS at 8 hpi. Two more such doses were given to hamsters in the following day. Since SARS-CoV-2 replicated quickly in hamster lungs and reached the peak titers at 2 dpi (J. F. Chan, et al., Clin Infect Dis, (2020); S. J. F. Kaptein, et al., Proc Natl Acad Sci U S A, 117 (2020) 26955-26965), the first dose was started at 8 hpi and viral loads in lungs were measured at 2 dpi (FIG. 4C). 4H30 (0.5 mg kg⁻¹ and 0.1 mg kg⁻¹) could significantly inhibit SARS-CoV-2 replication when compared with mock-treated hamsters.

To investigate the efficacy of 4H30 against SARS-CoV-2 variants, it was shown that 4H30 significantly inhibited the replication of four SARS-CoV-2 variants in cells (FIG. 4D). It was further confirmed that 4H30 (0.15 μmol kg⁻¹=0.5 mg kg⁻¹) significantly inhibited SARS-CoV-2 (B.1. 617.2, Delta) replication in hamsters (FIG. 4E), which was comparable to the effect of remdesivir with the dosage (0.75 μmol kg⁻¹=0.5 mg kg⁻¹). 4H30 reduced the syncytium formation and bronchial epithelial pathology in hamster lungs when compared with mock treated hamsters. However, syncytia were still observed in the bronchi of hamster lungs treated by remdesivir, which was consistent with the antiviral mechanism of remdesivir which interferes with RNA synthesis without affecting spike induced cell fusion. The reduced syncytia may reduce the damage of SARS-CoV-2 infection in vivo because patients who died from severe COVID-19 had syncytial changes in the lungs at autopsy (L. Braga, et al., Nature, 594, pages 88-93 (2021)). These results indicated that inhaled 4H30 (the dose of 0.5 mg kg⁻¹ in hamsters was less than the dose of 5 mg/50 kg in humans according to the guidance of Human Equivalent Dose suggested by FDA) could broadly inhibit viral replication of SARS-CoV-2 variants in hamsters with reduced syncytial formation in lungs.

### 4H30 Blocks SARS-CoV-2 Transmission

SARS-CoV-2 Delta variant was inoculated to hamsters which were treated by PBS or 4H30. Then naïve contact hamsters were put in the same cages separated by a plastic board to allow the airborne transmission. After 6 h exposure, the naïve contact hamsters were put in a new cage for viral culture. The lung and nasal turbinate tissues were collected at day 3 post-exposure for measuring viral loads by RT-qPCR and plaque assay. The PBS-treated hamsters showed 100% transmission in six hamsters ( 6/6), which indicated the high transmission potential of Delta variant in the airborne transmission in hamsters. 4H30-treated hamsters showed 25% transmission in 4 hamsters (¼), which indicated that 4H30 could block the transmission of SARS-CoV-2 in hamsters.

### 4H30 Inhibits H1N1 Virus Replication

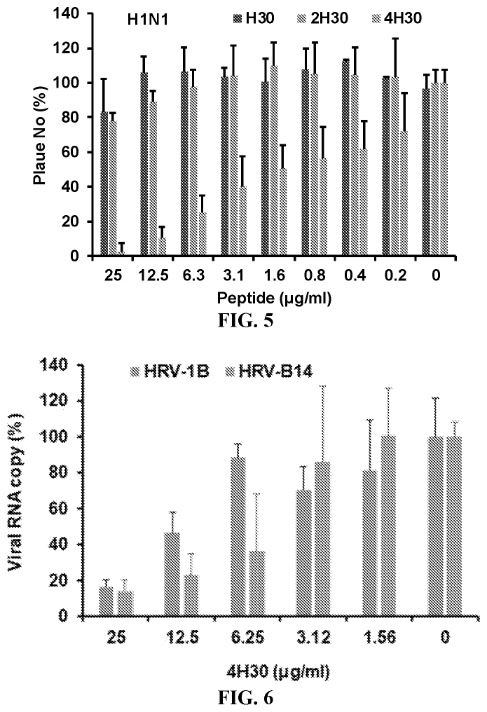

H1N1 virus was treated by hBD2 peptide H30, 2-branced H30 (2H30) or 4-branched H30 (4H30) for the plaque reduction assay. H1N1 virus was treated by hBD2 peptide H30, 2-branced H30 (2H30) or 4-branched H30 (4H30) in PBS for 30 min, and then the virus was inoculated to VeroE6 for the plaque reduction assay. H1N1 treated by PBS (0) was the no inhibition control. After 1 h incubation at 37° C. for viral infection, the un-infected virus was removed and 1% low melting agar was added to infected cells for forming plaque. After 3-day incubation at 37° C. The cells were fixed for counting the plaque number. Data are presented as mean±SD of at least three biological samples with more than two independent experiments. As shown in FIG. 5, H1N1 treated by PBS (0 μg/ml peptide) was the no inhibition control, H1N1 virus replication was significantly inhibited by 4H30.

### 4H30 Inhibits Minor Group HRV-1B and Major Group HRV-B14

Viruses were treated by 4H30 for infection in RD or H1-Hela cells. Viruses, HRV-1B and HRV-B14 were treated by 4H30 in PBS for 30 min and then the treated viruses were added to RD or H1-Hela cells for infection. After 1 h infection, the un-infected viruses were removed and fresh media with the indicated concentration of 4H30 was added to cells for viral culture. The supernatant viruses were measured at 30 hpi by RT-qPCR. Viral RNA copy (%) was the viral RNA of 4H30-treated virus normalized to that of untreated virus (0). Data are presented as mean±SD of at least three independent biological samples. The primers for detecting the viral RNA copies: HRV-F1: 5′-AGCCYGCGTGGCKGCC-3′; HRV-F2: 5′-AGCCYGCGTGGTGCCC-3′, HRV-R: 5′-GAAACACGGACACCCAAAGTAGT-3′; HRV-Probe: 5′ HEX-TCCGGCCCCTGAATGYGGCTAA-1ABKFQ 3′ (Y=C or T, K=G or T). The supernatant viruses were measured at 30 hpi. 4H30 effectively inhibited minor group HRV-1B and major group HRV-B14 (FIG. 6).

### Discussion

In this study, a human-sourced defensin peptide 4H30 which cross-linked SARS-CoV-2 to block viral entry outside the cell membrane of VeroE6 and Calu-3 cells was identified. It also inhibited viral entry through endocytic pathway by preventing endosomal acidification and inhibits SARS-CoV-2 release by cross-linking the viral spike with cell-surface GAGs. Thus, 4H30 provides a layer of protection on cell surface to block both viral entry and release to suppress viral infection and dissemination. Moreover, 4H30 inhibited spike-ACE2 mediated cell-cell fusion, which reduced syncytial formation indicating severe SARS-CoV-2 pneumonia. Importantly, 4H30 effectively inhibited SARS-CoV-2 variants in vitro and in hamsters, which implicated its broad antiviral activity, underscoring the clinical relevance of this human-sourced peptide.

With the circulation of pandemic SARS-CoV-2, variants with potential ability to escape from neutralizing antibody induced by vaccines are now important concerns. There is an urgency to find new antivirals with broad range of activities against coronaviruses. In this study, a human-sourced defensin peptide 4H30 was identified which contains the same segment of amino acid sequence found in the original human beta defensin 2. Thus 4H30 is expected to be safe for clinical trials. Recent studies reported that human beta defensin 2 could bind to spike protein to inhibit spike binding to ACE2 (L. Zhang, et al., bioRxiv, (2021)). However, the low antiviral activity of defensins in physiological salt condition and the difficulty to produce enough recombinant hBD2 might be an important barrier to manufacture it for clinical treatment. In addition, previous studies showed that hBD2 could bind to GAGs (E. S. Seo, et al., Biochemistry, 49 (2010) 10486-10495), but the antiviral activity of hBD2 is poor and little in vivo data are available (C. Xu, et al., Viruses, 13 (2021)). According to previous studies (30 amino acid P9 and P9R) related to mouse beta defensin 4 (the ortholog of HBD2) (H. Zhao, et al., Nat Commun, 12 (2021) 1517; H. Zhao, et al., Sci Rep, 6 (2016) 22008; H. Zhao, et al., Nat Commun, 11 (2020) 4252), 30 amino acid peptide H30 was designed from HBD2 and its antiviral activity was enhanced by making the branched 4H30. Thus, 4H30 was able to bind to SARS-CoV-2 spike and cross-link viral particles to block virus entry in both VeroE6 and Calu-3 cells. In addition, basic 4H30 inhibited endosomal acidification to block spike-ACE2 mediated fusion, similar to endosomal acidification inhibition of bafilomycin A1 and chloroquine to block spike-ACE2 mediated fusion (H. Zhao, et al., Nat Commun, 12 (2021) 1517). The inhibition on spike-ACE2 mediated cell fusion could be attributed to the reduction of syncytia, which was correlated with the severity of SARS-CoV-2 pneumonia (Z. Xu, et al., Lancet Respir Med, 8 (2020) 420-422). Since spike expression on infected cell surface can trigger the cell fusion with ACE2-expressed cells, the blocking cell-cell fusion by 4H30 might play an important role in reducing the syncytial formation and lung damage. Moreover, it has been showed that 4H30 cross-linked spike with cell surface GAGs (CS and HS) to block viral release, which could restrict the spreading of SARS-CoV-2 to non-infected cells. Thus, this discovery provides the evidence and strategy to inhibit SARS-CoV-2 release. Collectively, a 4-branched H30 (4H30) has been identified that binds spike and also GAGs which effectively cluster viral particles on cell membrane to block viral entry and release of newly synthesized virus.

More importantly, 4H30 showed potent antiviral activity against SARS-CoV-2 variants in vitro and in hamsters with the low dose (0.5 mg kg⁻¹), which may translate into promising antiviral activity in patients if administrated by the aerosol route. The inhaled vaccines also provided evidence that the aerosol inhalation of antivirals might be an alternative for treating COVID-19 (D. An, et al., Sci Adv, 7 (2021); S. Wu, et al., Lancet Infect Dis, (2021)). The discovery of this 4H30 with triple antiviral mechanisms (cross-linking viral particles, blocking cell-to-cell fusion, and inhibiting viral release) provides another antiviral strategy for the discovery of other antivirals with multiple antiviral mechanisms.

1. An antiviral peptide the sequence of any one of SEQ ID NO:4, SEQ ID NO:2 or SEQ ID NO:3, or is a variant having a sequence similarity of about 80%, 85%, 90%, 95% of any one of SEQ ID NO:4, SEQ ID NO:2 or SEQ ID NO:3.

2-3. (canceled)

4. A multimer comprising two or more antiviral peptides, wherein the antiviral peptides comprise an amino acid sequence that has the sequence SEQ ID NO:1 or is a fragment or variant of SEQ ID NO:1,
optionally wherein the amino acid sequence comprises a sequence similarity of about 80%, 85%, 90%, 95%, 99% to SEQ ID NO:1.

5. (canceled)

6. The multimer of claim 4, wherein the amino acid sequence comprises the sequence of any one of SEQ ID NO:4, SEQ ID NO:2 or SEQ ID NO: 3, or is a variant having a sequence similarity of about 80%, 85%, 90%, 95%, 99% of any one of SEQ ID NO:4, SEQ ID NO:2 or SEQ ID NO:3.

7. (canceled)

8. The multimer of claim 4, wherein the multimer is a dimer, trimer or tetramer, and/or is homomultimeric or heteromultimeric.

9. The multimer of claim 4, wherein the multimer is formed by:
(i) cross-linking each monomeric antiviral peptide, preferably using 2,2 bis(hydroxymethyl) propionic acid (MPA) or a 2nd generation MPA dendron with 4 reactive sites; or
(ii) coupling a MPA to two monomeric antiviral peptides comprising SEQ ID NO:4, cross-linked by lysine at C terminal to form a molecule with two branches of the antiviral peptide or by coupling a 2nd generation MPA dendron with 4 reactive sites to monomeric antiviral peptide having SEQ ID NO:4 cross-linked by lysine at C terminal to form a molecule with four branches of the antiviral peptide.

10. (canceled)

11. A composition comprising an antiviral peptide,
wherein the antiviral peptide comprises an amino acid sequence of any one of SEQ ID NO:1, SEQ ID NO:2, SEQ ID NO:3, or SEQ ID NO:4, or a fragment or variant thereof having a sequence similarity of about 80%, 85%, 90%, 95%, 99% to SEQ ID NO: 1, SEQ ID NO:2, SEQ ID NO:3, or SEQ ID NO: 4,
optionally wherein the composition further comprises one or more further therapeutic, prophylactic, or diagnostic agent.

12-13. (canceled)

14. The composition of claim 11, wherein the further agent is selected from a group consisting of bronchodilators, corticosteroids, methylxanthines, phosphodiesterase-4 inhibitors, anti-angiogenesis agents, antimicrobial agents, antioxidants, anti-inflammatory agents, immunosuppressant agents, anti-allergic agents, and combinations thereof.

15-17. (canceled)

18. The composition of claim 11, wherein two or more of the antiviral peptides are in a form of dimers, trimers, tetramers, or multimers.

19. The composition of claim 18, wherein the antiviral peptide comprises SEQ ID NO:4 in a form of tetramers.

20. A pharmaceutical composition comprising the antiviral peptide of claim 1, and a pharmaceutically acceptable carrier.

21. The pharmaceutical composition of claim 20, wherein the composition is lyophilized or the composition is in a form selected from a group consisting of aerosol powder, liquids, and suspensions.

22. A kit comprising
(a) one or more single unit dose of a composition comprising the antiviral peptide of claim 1, and
(b) instructions on how the dose is to be administered for treatment or prevent of coronavirus infection, influenza virus infection or rhinovirus infection.

23. A method of treating or retarding the development of one or more symptoms of respiratory viral infections or blocking virus transmission comprising administering to a subject in need thereof an effective amount of the antiviral peptide of the composition of claim 1,
optionally wherein the composition is administered in combination with another therapeutic, prophylactic, or diagnostic agent.

24. The method of claim 23, wherein the subject has a respiratory viral infection or at risk of contracting a respiratory virus,
optionally wherein the respiratory virus is selected from a group consisting of SARS-CoV-2 virus, influenza virus or rhinovirus.

25. (canceled)

26. The method of claim 24, wherein the influenza virus is HIN1 virus and/or the rhinovirus is HRV-1B or HRV-B14.

27. The method of claim 23, wherein the respiratory virus is a SARS-CoV-2 virus,
optionally wherein the virus is a SARS-CoV-2 virus variant selected from the group consisting of SARS-CoV-2 B.1.1.7 (Alpha variant), SARS-CoV-2 B.1.351 (Beta variant), SARS-CoV-2 P.1 (Gamma variant), SARS-CoV-2 B.1.617, SARS-CoV-2 B.1.617.1 (Kappa variant), SARS-CoV-2 B.1.621 (Mu variant), SARS-CoV-2 B.1.617.2 (Delta variant), SARS-CoV-2 B.1.617.3, and SARS-CoV-2 B.1.1.529 (Omicron variant).

28. The method of claim 23, wherein the composition is administered to the pulmonary or nasal system,
optionally wherein the composition is administered via a nebulizer or an inhaler.

29. The method of claim 23, wherein the composition is administered in a form selected from the group consisting of powder, liquids, aerosol and suspensions.

30-32. (canceled)

33. The method of claim 23, wherein the composition is administered in combination with one or more agents selected from the group consisting of bronchodilators, corticosteroids, methylxanthines, phosphodiesterase-4 inhibitors, anti-angiogenesis agents, antimicrobial agents, antioxidants, anti-inflammatory agents, immunosuppressant agents, anti-allergic agents, and combinations thereof.

34. The method of claim 23, wherein the composition is administered more than once, and
wherein the interval between doses is selected from the group consisting of once a week, once every two weeks, approximately once a month, once every two months and once every three months,
optionally wherein the composition is administered once a week for up to a period of 1, 2, 3, 4, 5, or 6 months.

35. (canceled)

36. The method of claim 23, wherein the composition is administered to a human subject at a dose of between 0.001 mg/kg body weight of the subject and 100 mg/kg body weight of the subject, inclusive, or at a dose of between 2.0 mg and 20 mg, inclusive, or at a dose of 5 mg.

37-38. (canceled)

39. The method of claim 23, wherein the composition is administered in an amount effective to:
(i) reduce syncytial formation and lung damage in the subject, and/or
(ii) reduce one or more symptoms of cough, fatigue, fever, body aches, headache, sore throat, loss or altered sense of taste and/or smell, vomiting, diarrhea, cytokine storm, skin changes, ocular complications, confusion, chronic neurological impairment, chest pain and shortness of breath.

40. (canceled)


SEQUENCE LISTING (10 sequences)

SEQ ID NO: 1 - protein, 64 aa, synthetic construct
MRVLYLLFSFLFIFLMPLPGVFGGIGDPVTCLKSGAICHPVFCPRRYKQIGTCGLPGTKCCKKP

SEQ ID NO: 2 - protein, 23 aa, synthetic construct
CHPVFCPRRYKQIGTCGLPGTKC

SEQ ID NO: 3 - protein, 26 aa, synthetic construct
CHPVFCPRRYKQIGTCGLPGTKCCKK

SEQ ID NO: 4 - protein, 30 aa, synthetic construct
GAICHPVFCPRRYKQIGTCGLPGTKCCKKP

SEQ ID NO: 5 - DNA, 30 bp, synthetic construct
  source 1..30: mol_type = other DNA
cctactaaattaaatgatctctgctttact

SEQ ID NO: 6 - DNA, 21 bp, synthetic construct
  source 1..21: mol_type = other DNA
caagctataacgcagcctgta

SEQ ID NO: 7 - DNA, 16 bp, synthetic construct
  source 1..16: mol_type = other DNA
agccygcgtggckgcc

SEQ ID NO: 8 - DNA, 16 bp, synthetic construct
  source 1..16: mol_type = other DNA
agccygcgtggtgccc

SEQ ID NO: 9 - DNA, 23 bp, synthetic construct
  source 1..23: mol_type = other DNA
gaaacacggacacccaaagtagt

SEQ ID NO: 10 - DNA, 22 bp, synthetic construct
  source 1..22: mol_type = other DNA
tccggcccctgaatgyggctaa